(19) United States
(12) Patent Application Publication
(10) Pub. No.: US 2025/0075277 A1
(43) Pub. Date: Mar. 6, 2025

(54) METHODS OF DETECTING ONCOGENIC FUSIONS AND USES THEREOF

(71) Applicant: Washington University, St. Louis, MO (US)

(72) Inventors: Andrew Young, St. Louis, MO (US); Hannah Davis, St. Louis, MO (US); Grant Challen, St. Louis, MO (US)

(73) Assignee: Washington University, St. Louis, MO (US)

(21) Appl. No.: 18/818,523

(22) Filed: Aug. 28, 2024

Related U.S. Application Data
(60) Provisional application No. 63/579,041, filed on Aug. 28, 2023.

(51) Int. Cl. C12Q 1/6886; C12N 15/10; C12Q 1/6806; C12Q 1/686; G01N 21/64

(52) U.S. Cl. CPC C12Q 1/6886; C12N 15/1096; C12Q 1/6806; C12Q 1/686; G01N 21/6486; C12Q 2539/105; C12Q 2600/106; C12Q 2600/156

(57) ABSTRACT

Among the various aspects of the present disclosure is the provision of a ddPCR-based method of detecting oncogenic fusions, and its use in screening and monitoring a cancer treatment is disclosed. A method for detecting oncogenic fusions involving the histone-lysine N-methyltransferase 2A (KMT2A) gene is described.

### CROSS-REFERENCE TO RELATED APPLICATIONS

This application claims priority from U.S. Provisional Application Ser. No. 63/579,041 filed on Aug. 28, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide sequences of the present invention. The subject matter of the Sequence Listing is incorporated herein by reference in its entirety.

### FIELD OF THE INVENTION

The present disclosure generally relates to methods of detecting oncogenic fusions.

### BACKGROUND OF THE INVENTION

Acute myeloid leukemia (AML) is an aggressive blood cancer that is driven by a diverse, but finite set of known oncogenic drivers. Detecting persistent leukemic cells after treatment is essential for selecting subsequent treatment and long-term prognostication. Currently, the methods for detecting measurable residual disease (MRD) after treatment for AML include bone marrow morphology, multiparameter flow cytometry (MPFC), and DNA sequencing. Morphologic assessment only detects leukemic cells at a 5% limit of detection. MPFC has a more sensitive limit of detection to 0.01-0.001% but is challenging to interpret and not standardized between labs. DNA sequencing approaches can identify leukemic cells by their somatic mutation profile but are expensive assays to run and can be confounded by clonal hematopoiesis in non-leukemic blood cells. For the subset of AML patients with oncogenic fusions driving their disease, the fusion itself is a molecular marker that can be leveraged for sensitive measurable residual disease detection.

Oncogenic fusions are currently utilized for disease monitoring in hematologic malignancies such as chronic myeloid leukemia (CML). The BCR-ABL1 fusion is the canonical driver mutation in CML. Reverse-transcription quantitative polymerase chain reaction (RT-qPCR) sensitively identifies BCR-ABL1 fusions from the mRNA transcript by utilizing primers that span the fusion breakpoint to a limit of detection of 1 in 100,000 cells. Response to therapy is assessed by log-order decreases in transcript abundance measured by RT-qPCR with a 10-3 BCR-ABL1 abundance classified as a major molecular response (MR3), and 10-4.5 (MR4.5)-105 (MR5) BCR-ABL1 abundance marking a deep molecular response and the limit of detection for most available assays. As tyrosine-kinase inhibitors have become more efficacious, individuals who clear their CML as assessed by RT-qPCR can discontinue therapy and about half of these individuals remain disease-free long-term. While BCR-ABL1 is almost universally associated with CML, there is no similar singular oncogenic fusion associated with AML.

The most common translocations associated with de novo AML, including RUNX1-RUNX1T1, CBFB-MYH11, and PML-RARA, can be detected with qPCR-based assays. Droplet digital PCR (ddPCR) improves upon qPCR by partitioning each reaction into a microfluid droplet enabling absolute quantification of nucleic acids in a sample. The improvements of ddPCR over qPCR assay are ease of implementation, improved lower limit of detection, high specificity, and absolute quantification (compared to relative quantification with a standard curve in qPCR). Already, ddPCR has demonstrated utility for the detection of BCR-ABL1 fusions associated with CML and PML-RARA fusions associated with acute promyelocytic leukemia. This platform has the potential to improve the ability to detect rare AML driven by chromosomal translocations. However, these techniques are difficult to implement when the gene fusion involves many different partners; such fusions involving multiple partners are not detectable with a single assay.

Therapy-related AML (t-AML) is a uniquely aggressive and treatment-resistant subpopulation of AML that arises after antecedent chemotherapy or radiation exposure, typically following the treatment of solid tumors. Prior work has demonstrated that cytotoxic therapy can select for pre-existing pre-malignant hematopoietic stem and progenitor cells leading to t-AML. In other cases, the therapy itself creates the oncogenic initiating event, such as the introduction of double-strand DNA breaks. One class of chemotherapy—topoisomerase II (TOP2) inhibitors—is uniquely associated with oncogenic fusions involving the histone-lysine N-methyltransferase 2A (KMT2A) gene. There are at least 80 known KMT2A fusion partners, but about 80% of fusions involve just five partners—AF9, AF6, AF4, ELL, and ENL. These fusions are potent drivers of leukemia. Mouse models show that the introduction of these fusions into healthy bone marrow progenitor cells can drive an aggressive AML as a singular event. Moreover, pediatric leukemia driven by KMT2A fusions often arises without any cooperating mutations. The development of t-AML in pediatric cancer patients who receive high doses of TOP2 inhibitor therapy is a devastating consequence of the treatment of their primary malignancy that is almost universally fatal. The ability to sensitively detect KMT2A fusions arising in hematopoietic cells during the course of therapy or in remission could identify such patients at increased risk of t-AML and stratify them for intervening measures.

### SUMMARY OF THE INVENTION

Among the various aspects of the present disclosure is the provision of a method for detecting at least one oncogenic fusion in a subject.

In one aspect, a method for monitoring an abundance of expression of at least one oncogenic KMT2A fusion in a subject is described, in which each KMT2A fusion includes a KMT2A fragment fused at a fusion site to a partner gene fragment. The method includes providing a biological sample from the subject that includes an amount of RNA; at least one forward PCR primer targeting a KMT2A start codon of the KMT2A fragment upstream of the fusion site and at least one reverse PCR primer targeting a partner gene start codon of the partner gene fragment downstream of the fusion site; at least one KMT2A probe that includes a first fluorescent reporter, wherein each KMT2A probe is configured to anneal to the KMT2A fragment between the KMT2A start codon and the fusion site; and at least one partner gene probe that includes a second fluorescent reporter, wherein each partner gene probe is configured to anneal to the partner gene fragment between the partner start codon and the fusion site. The method further includes extracting the amount of RNA from the biological sample and synthesizing an amount of cDNA from the amount of RNA; subjecting a mixture of the amount of cDNA, the at least one forward PCR primer, the at least one reverse PCR primer, the at least one KMT2A probe, and the at least one partner gene probe to droplet digital PCR (ddPCR) to obtain a plurality of paired fluorescence intensities from first and second reporters; and estimating the abundance of expression of the at least one oncogenic KMT2A fusion based on the plurality of paired fluorescence intensities obtained from the ddPCR. In some aspects, the first and second fluorescent reporters are independently selected from fluorescein and hexachlorofluorescein, wherein the first fluorescent reporter is different from the second fluorescent reporter. In some aspects, the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof. In some aspects, the at least one oncogenic KMT2A fusion is one KMT2A fusion from the group consisting of KMT2A-AF9, KMT2A-AF4, KMT2A-AF6, KMT2A-ENL, and KMT2A-ELL. In some aspects, the at least one forward PCR primer targets the KMT2A start codon of the KMT2A fragment selected from KMT2A exon 7, KMT2A exon 9, and any combination thereof; and the at least one reverse PCR primer targets the partner start codon of the partner gene fragment selected from AF9 exon 6, AF4 exon 5, AF6 exon 2, ENL exon 7, ELL exon 3, and any combination thereof. In some aspects, the at least one forward PCR primer includes a nucleotide sequence selected from SEQ ID NO:1 targeting KMT2A exon 7, SEQ ID NO:2 targeting KMT2A exon 9, and any combination thereof; and the at least one reverse PCR primer includes a nucleotide sequence selected from SEQ ID NO:7 targeting AF9 exon 6; SEQ ID NO:5 targeting AF4 exon 5; SEQ ID NO:9 targeting AF6 exon 2; SEQ ID NO:13 targeting ENL exon 7; SEQ ID NO:14 targeting ELL exon 3; and any combination thereof. In some aspects, the at least one KMT2A probe includes a nucleotide sequence selected from SEQ ID NO:16 targeting KMT2A exon 7, SEQ ID NO:22 targeting KMT2A exon 9, and any combination thereof. In some aspects, the at least one partner gene probe includes a nucleotide sequence selected from SEQ ID NO:18 targeting AF9 exon 6; SEQ ID NO:17 targeting AF4 exon 5; SEQ ID NO:19 targeting AF6 exon 2; SEQ ID NO:21 targeting ENL exon 7; SEQ ID NO:23 targeting ELL exon 3; and any combination thereof. The sample includes at least one of a peripheral blood sample, a bone marrow sample, a solid tumor sample, and any combination thereof. In some aspects, the method further includes identifying at least one subject-specific KMT2A fusion from a genomic sequencing of the subject's leukemia, wherein each subject-specific KMT2A fusion includes a KMT2A fragment fused at a fusion site to a subject-specific partner gene fragment, wherein the at least one reverse PCR primer further includes an additional reverse PCR primer targeting a subject-specific partner gene start codon of the subject-specific partner gene fragment downstream of the fusion site; and the at least one partner gene probe further includes at least one subject-specific partner gene probe including the second fluorescent reporter, wherein each subject-specific partner gene probe is configured to anneal to the subject-specific partner gene fragment between the subject-specific partner gene start codon and the fusion site. In some aspects, the method further includes classifying the cancer patient as having measurable residual disease if the abundance of expression of the at least one oncogenic KMT2A fusion falls above a threshold value of 0.001% as measured by ddPCR for the oncogenic KMT2A fusion. In some aspects, the method further includes providing first and second biological samples from the subject, wherein the second sample is obtained after administration of a treatment to the subject; estimating a first and second abundance of expression of the at least one oncogenic KMT2A fusion based on the first and second biological samples; estimating an efficacy of the treatment, a development of treatment-related oncogenic KMT2A fusions, a prognosis, and any combination thereof based on changes between the first and second abundances of expression of the at least one oncogenic KMT2A fusion.

In another aspect, a method for personalized monitoring an abundance of expression of at least one oncogenic KMT2A fusion in a patient, in which each KMT2A fusion includes a KMT2A fragment fused at a fusion site to a partner gene fragment is disclosed. The method includes providing an initial sequencing of the patient's leukemia at a nucleotide-level resolution and identifying at least one oncogenic KMT2A fusion based on the initial sequencing. The method further includes providing a biological sample from the patient that includes an amount of RNA; at least one forward PCR primer targeting a KMT2A start codon of the KMT2A fragment upstream of the fusion site and at least one reverse PCR primer targeting a partner gene start codon of the partner gene fragment downstream of the fusion site; at least one KMT2A probe including a first fluorescent reporter, wherein each KMT2A probe is configured to anneal to the KMT2A fragment between the KMT2A start codon and the fusion site; and at least one partner gene probe including a second fluorescent reporter, wherein each partner gene probe is configured to anneal to the partner gene fragment between the partner start codon and the fusion site. The method further includes extracting the amount of RNA from the biological sample and synthesizing an amount of cDNA from the amount of RNA; subjecting a mixture of the amount of cDNA, the at least one forward PCR primer, the at least one reverse PCR primer, the at least one KMT2A probe, and the at least one partner gene probe to droplet digital PCR (ddPCR) to obtain a plurality of paired fluorescence intensities from first and second reporters; and estimating the abundance of expression of the at least one oncogenic KMT2A fusion based on the plurality of paired fluorescence intensities obtained from the ddPCR.

In another aspect, an assay to monitor an abundance of expression of at least one oncogenic KMT2A fusion in a subject, in which each KMT2A fusion including a KMT2A fragment fused at a fusion site to a partner gene fragment is disclosed. The assay includes at least one forward PCR primer targeting a KMT2A start codon of the KMT2A fragment upstream of the fusion site and at least one reverse PCR primer targeting a partner gene start codon of the partner gene fragment downstream of the fusion site; at least one KMT2A probe including a first fluorescent reporter, wherein each KMT2A probe is configured to anneal to the KMT2A fragment between the KMT2A start codon and the fusion site; and at least one partner gene probe including a second fluorescent reporter, wherein each partner gene probe is configured to anneal to the partner gene fragment between the partner start codon and the fusion site. The at least one forward PCR primer, the at least one reverse PCR primer, the at least one KMT2A probe, and the at least one partner gene probe are combined with an amount of cDNA synthesized from an amount of RNA from the subject to form a mixture for processing in a droplet digital PCR (ddPCR) device. In some aspects, the first and second fluorescent reporters are independently selected from fluorescein and hexachlorofluorescein, wherein the first fluorescent reporter is different from the second fluorescent reporter. In some aspects, the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof. In some aspects, the at least one oncogenic KMT2A fusion is one KMT2A fusion from the group consisting of KMT2A-AF9, KMT2A-AF4, KMT2A-AF6, KMT2A-ENL, and KMT2A-ELL. In some aspects, the at least one forward PCR primer targets the KMT2A start codon of the KMT2A fragment selected from KMT2A exon 7, KMT2A exon 9, and any combination thereof; and the at least one reverse PCR primer targets the partner start codon of the partner gene fragment selected from AF9 exon 6, AF4 exon 5, AF6 exon 2, ENL exon 7, ELL exon 3, and any combination thereof. In some aspects, the at least one forward PCR primer includes a nucleotide sequence selected from SEQ ID NO:1 targeting KMT2A exon 7, SEQ ID NO:2 targeting KMT2A exon 9, and any combination thereof; and the at least one reverse PCR primer includes a nucleotide sequence selected from SEQ ID NO:7 targeting AF9 exon 6; SEQ ID NO:5 targeting AF4 exon 5; SEQ ID NO:9 targeting AF6 exon 2; SEQ ID NO:13 targeting ENL exon 7; SEQ ID NO:14 targeting ELL exon 3; and any combination thereof. In some aspects the at least one KMT2A probe includes a nucleotide sequence selected from SEQ ID NO:16 targeting KMT2A exon 7, SEQ ID NO:22 targeting KMT2A exon 9, and any combination thereof. In some aspects, the at least one partner gene probe includes a nucleotide sequence selected from SEQ ID NO:18 targeting AF9 exon 6; SEQ ID NO:17 targeting AF4 exon 5; SEQ ID NO:19 targeting AF6 exon 2; SEQ ID NO:21 targeting ENL exon 7; SEQ ID NO:23 targeting ELL exon 3; and any combination thereof.

In another aspect, a kit for monitoring an abundance of expression of at least one oncogenic KMT2A fusion in a subject, in which KMT2A fusion includes a KMT2A fragment fused at a fusion site to a partner gene fragment. The kit includes at least one forward PCR primer targeting a KMT2A start codon of the KMT2A fragment upstream of the fusion site and at least one reverse PCR primer targeting a partner gene start codon of the partner gene fragment downstream of the fusion site; at least one KMT2A probe including a first fluorescent reporter, wherein each KMT2A probe is configured to anneal to the KMT2A fragment between the KMT2A start codon and the fusion site; and at least one partner gene probe including a second fluorescent reporter, wherein each partner gene probe is configured to anneal to the partner gene fragment between the partner start codon and the fusion site. In some aspects, the first and second fluorescent reporters are independently selected from fluorescein and hexachlorofluorescein, wherein the first fluorescent reporter is different from the second fluorescent reporter. In some aspects, the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof. In some aspects, the at least one oncogenic KMT2A fusion is one KMT2A fusion from the group consisting of KMT2A-AF9, KMT2A-AF4, KMT2A-AF6, KMT2A-ENL, and KMT2A-ELL. In some aspects, the at least one forward PCR primer targets the KMT2A start codon of the KMT2A fragment selected from KMT2A exon 7, KMT2A exon 9, and any combination thereof; and the at least one reverse PCR primer targets the partner start codon of the partner gene fragment selected from AF9 exon 6, AF4 exon 5, AF6 exon 2, ENL exon 7, ELL exon 3, and any combination thereof. In some aspects, the at least one forward PCR primer includes a nucleotide sequence selected from SEQ ID NO: 1 targeting KMT2A exon 7, SEQ ID NO:2 targeting KMT2A exon 9, and any combination thereof; and the at least one reverse PCR primer includes a nucleotide sequence selected from SEQ ID NO:7 targeting AF9 exon 6, SEQ ID NO: 5 targeting AF4 exon 5, SEQ ID NO:9 targeting AF6 exon 2, SEQ ID NO:13 targeting ENL exon 7, SEQ ID NO:14 targeting ELL exon 3, and any combination thereof. In some aspects, the at least one KMT2A probe includes a nucleotide sequence selected from SEQ ID NO:16 targeting KMT2A exon 7, SEQ ID NO:22 targeting KMT2A exon 9, and any combination thereof. In some aspects, the at least one partner gene probe includes a nucleotide sequence selected from SEQ ID NO: 18 targeting AF9 exon 6, SEQ ID NO:17 targeting AF4 exon 5, SEQ ID NO: 19 targeting AF6 exon 2, SEQ ID NO:21 targeting ENL exon 7, SEQ ID NO: 23 targeting ELL exon 3, and any combination thereof.

Additional aspects and embodiments of the method for detecting at least one oncogenic fusion in a subject are disclosed in detail herein.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic diagram describing primer and probe design to enable detection of KMT2A-AF4 fusions. Primers span the fusion breakpoints (KMT2A-AF4 depicted). Nested fluorescently labelled probes recognized unique sequences in KMT2A (FAM) and the fusion partner AFF1-AF4 (HEX).

FIG. 1B is a representative graph of ddPCR output containing double positive droplets marking the KMT2A fusion transcripts. The ddPCR result includes double positive droplets containing cDNA with an oncogenic KMT2A fusion (+). Double negative droplets contain wild-type KMT2A cDNA or no cDNA from KMT2A (−).

FIG. 2A is a graph wherein cell lines harboring 457 KMT2A fusions were serially diluted over six orders of magnitude into OCI-AML3 cells (KMT2A 458 wild-type). Oncogenic KMT2A fusions were detected using primer/probe pairs targeting the 459 cell-type specific KMT2A fusions.

FIG. 2B contains graphs summarizing droplet digital PCR results for HEL cells edited with CRISPR/Cas9 using guides targeting the KMT2A and AF9 loci. HEL cells with KMT2A-AF9 fusions were detected at days 4, 14 and 21 (top facets). Fusion transcript abundance was compared to wild-type KMT2A transcript abundance (bottom facets).

FIG. 2C is a quantification of KMT2A-AF9 fusions in edited HEL cells over time illustrated in FIG. 2B, shown normalized to wild-type KMT2A transcript abundance.

FIG. 3A contains graphs summarizing ddPCR results using pooled primer/probe pairs targeting the five most common KMT2A fusions (left facets). KMT2A fusions were detected in THP-1 cells (KMT2A-AF9}, but not detected in Kasumi cells (KMT2A wild-type). KMT2A wild-type transcript abundance is included for comparison.

FIG. 3B is a graph of KMT2A fusion transcript abundance from multiple cell lines known to harbor KMT2A fusions normalized to the KMT2A wild-type transcript abundance.

FIG. 3C is a graph of KMT2A fusion transcript abundance from multiple patient samples known to harbor KMT2A fusions normalized to the KMT2A wild-type transcript abundance.

FIG. 3D is a graph of a dilution series experiment over six orders of magnitude using the pooled primer/probe pairs targeting the five most common KMT2A fusions.

FIG. 4 is a table of primer and probe sequences for KMT2A fusion detection. All primers tested are listed. * Denotes primers moved forward for the ddPCR assay.

FIG. 5 is a table of single guide RNA molecules designed to introduce KMT2A-AF9 rearrangements using CRISPR/Cas9.

FIG. 6 is a set of UCSC Genome Browser (https://qenome.ucsc.edu/; last accessed Feb. 10, 2023) mapping results for cell line KMT2A fusions from DepMap (https://depmap.orq/portal/; last accessed Dec. 17, 2021), primer/probe sequences, and cell-line cDNA sequencing results. Gene annotations by NCBI RefSeq and Ensembl (last accessed Feb. 10, 2023).

FIG. 7 is a set of graphs of dilution series experiment conducted to benchmark the fusion-specific primer/probe pairs using qPCR. Primer/probe pair combinations targeting the KMT2A wild-type transcript were also included for comparison.

FIG. 8A is a set of graphs summarizing ddPCR results for a dilution series using the KOPN8 (KMT2A-ENL) cell line with known KMT2A fusions diluted into OCI-AML3 cells (KMT2A wild-type). KMT2A fusions were detected with primer/probe pairs designed to target the cell-type specific KMT2A fusions. Dilutions from top to bottom are 50%, 5%, 0.5%, 0.05%, 0.005% and 0.0005%. Horizontal and vertical lines demarcated the positive and negative cutoffs for fluorescent intensity.

FIG. 8B is a set of graphs summarizing ddPCR results for a dilution series using the MOLM13 (KMT2A-AF9) cell line with known KMT2A fusions diluted into OCI-AML3 cells (KMT2A wild-type).

FIG. 8C is a set of graphs summarizing ddPCR results for a dilution series using the MV4-11 KOPN8 (KMT2A-AF4) cell line with known KMT2A fusions diluted into OCI-AML3 cells (KMT2A wild-type).

FIG. 8D is a set of graphs summarizing ddPCR results for a dilution series using the OCI-AML2 (KMT2A-AF6) cell line with known KMT2A fusions diluted into OCI-AML3 cells (KMT2A wild-type).

FIG. 8E is a set of graphs summarizing ddPCR results for a dilution series using the THP-1 (KMT2A-AF9) cell line with known KMT2A fusions diluted into OCI-AML3 cells (KMT2A wild-type).

FIG. 9A is a set of graphs summarizing ddPCR results for individual KMT2A fusion primer/probe pairs designed to target KMT2A-AF9 as tested in cell lines harboring different KMT2A fusions to evaluate off-target amplification and probe binding.

FIG. 9B is a set of graphs summarizing ddPCR results for individual KMT2A fusion primer/probe pairs designed to target KMT2A-AF6, tested in cell lines harboring different KMT2A fusions to test off-target amplification and probe binding.

FIG. 9C is a set of graphs summarizing ddPCR results for individual KMT2A fusion primer/probe pairs designed to target KMT2A-AF4, tested in cell lines harboring different KMT2A fusions to test off-target amplification and probe binding.

FIG. 9D is a set of graphs summarizing ddPCR results for individual KMT2A fusion primer/probe pairs designed to target KMT2A-ELL, tested in cell lines harboring different KMT2A fusions to test off-target amplification and probe binding.

FIG. 9E is a set of graphs summarizing ddPCR results for individual KMT2A fusion primer/probe pairs designed to target KMT2A-ENL, tested in cell lines harboring different KMT2A fusions to test off-target amplification and probe binding.

FIG. 10A is a set of graphs summarizing ddPCR results for pooled primer/probe pairs for cell lines with KMT2A fusions, THP-1 and OCI-AML2. ddPCR results using pooled KMT2A fusion primers and probes (left) are compared to the KMT2A wild-type primer/probe pair (right). KMT2A fusions were not detected in the KMT2A wild-type cell lines (OCI-AML3, Kasumi, Jurkat).

FIG. 10B is a set of graphs summarizing ddPCR results for pooled primer/probe pairs for cell lines with KMT2A fusions, MV4-11 and MOL13. ddPCR results using pooled KMT2A fusion primers and probes (left) are compared to the KMT2A wild-type primer/probe pair (right).

FIG. 10C is a set of graphs summarizing ddPCR results for pooled primer/probe pairs for a cell line with KMT2A fusions, KOPN8. ddPCR results using pooled KMT2A fusion primers and probes (left) are compared to the KMT2A wild-type primer/probe pair (right).

FIG. 10D is a set of graphs summarizing ddPCR results for pooled primer/probe pairs for cell lines without KMT2A fusions, OCI-AML3 and Kasumi. Plots depicted ddPCR results using pooled KMT2A fusion primers and probes (left) are compared to the KMT2A wild-type primer/probe pair (right). KMT2A fusions were not detected in the KMT2A wild-type cell lines (OCI-AML3, Kasumi, Jurkat).

FIG. 10E is a set of graphs summarizing ddPCR results for pooled primer/probe pairs for a cell line without KMT2A fusions, Jurkat. Plots depicted ddPCR results using pooled KMT2A fusion primers and probes (left) are compared to the KMT2A wild-type primer/probe pair (right). KMT2A fusions were not detected in the KMT2A wild-type cell lines (OCI-AML3, Kasumi, Jurkat).

FIG. 11A is a set of graphs summarizing ddPCR results for two AML patient samples with known KMT2A fusions assayed with pooled primers and probes targeting the five most common KMT2A fusions. Plots depict ddPCR results using pooled KMT2A fusions primers and probes (left) compared to KMT2A wild-type primer/probe pair (right) as a control.

FIG. 11B is a set of graphs summarizing ddPCR results for two additional AML patient samples with known KMT2A fusions assayed with pooled primers and probes targeting the five most common KMT2A fusions. Plots depict ddPCR results using pooled KMT2A fusions primers and probes (left) compared to KMT2A wild-type primer/probe pair (right) as a control.

FIG. 11C is a set of graphs summarizing ddPCR results for AML patient samples without KMT2A fusions assayed with pooled primers and probes targeting the five most common KMT2A fusions. Plots depicted ddPCR results using pooled KMT2A fusions primers and probes (left) compared to KMT2A wild-type primer/probe pair (right) as a control.

FIG. 11D is a set of graphs summarizing ddPCR results for additional AML patient samples without KMT2A fusions assayed with pooled primers and probes targeting the five most common KMT2A fusions. Plots depicted ddPCR results using pooled KMT2A fusions primers and probes (left) compared to KMT2A wild-type primer/probe pair (right) as a control.

FIG. 12A is a set of graphs summarizing ddPCR results for a pooled primer/probe dilution series using KOPN8 (KMT2A-ENL) cell line with known KMT2A fusions diluted into OCI-AML3 cells (KMT2A wild-type). KMT2A fusions were detected with the pooled primers and probes designed to target the five most common KMT2A fusions.

FIG. 12B is a set of graphs summarizing ddPCR results for a pooled primer/probe dilution series using MOLM13 (KMT2A-AF9) cell line with known KMT2A fusions diluted into OCI-AML3 cells (KMT2A wild-type). KMT2A fusions were detected with the pooled primers and probes designed to target the five most common KMT2A fusions.

FIG. 12C is a set of graphs summarizing ddPCR results for a pooled primer/probe dilution series using MV4-11 (KMT2A-AF4) cell line with known KMT2A fusions diluted into OCI-AML3 cells (KMT2A wild-type). KMT2A fusions were detected with the pooled primers and probes designed to target the five most common KMT2A fusions.

FIG. 12D is a set of graphs summarizing ddPCR results for a pooled primer/probe dilution series using OCI-AML2 (KMT2A-AF6) cell line with known KMT2A fusions diluted into OCI-AML3 cells (KMT2A wild-type). KMT2A fusions were detected with the pooled primers and probes designed to target the five most common KMT2A fusions.

FIG. 12E is a set of graphs summarizing ddPCR results for a pooled primer/probe dilution series using THP-1 (KMT2A-AF9) cell line with known KMT2A fusions diluted into OCI-AML3 cells (KMT2A wild-type). KMT2A fusions were detected with the pooled primers and probes designed to target the five most common KMT2A fusions.

FIG. 13 is a table summarizing the mixture of pooled primers and probes used to produce 10× concentrated master mix. The final master mix volume was 200 μL. The stock primer and probes were 100 uM, each primer was 1000 nM, and each probe was 250 nM in the final 20 ul ddPCR reaction.

FIG. 14 is a table summarizing ddPCR results for a dilution series with fusion-specific primer/probes. Fusion transcript concentration and wild-type KMT2A transcript concentration for dilution series experiments using cell lines with known KMT2A fusions diluted into OCI-AML3 cells. Fusions were assayed with the individual primer/probe pair designed to detect that fusion.

FIG. 15 is a table summarizing ddPCR results for samples from several patients with KMT2A fusion-driven leukemia. The KMT2A fusion transcript abundance measured by ddPCR is shown compared to standard pathological assessments of leukemia burden and KMT2A fusion abundance, including fluorescence in situ hybridization (FISH), cytogenetics, whole genome sequence (WGS), fraction of leukemic blasts by morphological assessment and fraction of leukemic blasts by flow cytometry. ND denotes not detected, and N/A denotes not applicable/was not assessed.

FIG. 16 is a table summarizing ddPCR results for a dilution series with pooled primer/probes. Fusion transcript concentration and wild-type KMT2A transcript concentration for dilution series experiments using cell lines with known KMT2A fusions were diluted into OCI-AML3 cells. Fusions were assayed with the pooled primer/probe pairs of FIG. 13 to detect a panel of KMT2A fusions.

### DETAILED DESCRIPTION OF THE INVENTION

In various aspects, a ddPCR assay for the detection of oncogenic fusions is disclosed. In one exemplary aspect, a ddPCR assay is disclosed that detects the five most common KMT2A fusions accounting for the vast majority (approximately 80%) of oncogenic KMT2A fusions found in treatment-related acute myeloid leukemia (t-AML) is disclosed herein. In various aspects, use of the disclosed ddPCR assay improves disease detection and treatment decision-making for t-AML patients with KMT2A fusions and detects pre-malignant oncogenic fusions in at-risk individuals after chemotherapy exposure.

The disclosed assay builds upon prior work detecting multiple oncogenic fusions in Ewing's Sarcoma and to improve detection for a variety of other cancers including, but not limited to, fusion-driven liquid cancers. As described in the examples, the disclosed assay was benchmarked using cell lines and primary patient samples with known KMT2A fusions. The disclosed assay is an inexpensive, rapid, easy to implement, sensitive, specific, and easily interpreted platform for the detection and quantification of KMT2A fusion and other oncogenic fusions that improves the detection of measurable residual disease detection for a variety of patients including, but not limited to, AML patients with KMT2A fusions.

The disclosed method makes use of droplet digital PCR (ddPCR) technology to identify oncogenic fusions and overcomes at least some of the limitations of existing oncogenic fusion detections methods, such as RT-qPCR methods. The use of ddPCR as a detection modality obviates the need for standard curve calibration used in previous methods because ddPCR quantitation is absolute. Prior studies have reported qPCR and ddPCR assays for fusion detection in CML (BCR-ABL1), but not for AML. In some aspects, the disclosed assay is suitable for the detection of a variety of oncogenic fusions associated with a variety of cancers including, but not limited to, oncogenic KMT2A fusions, one of the most common therapy-related AML translocations.

Previous oncogenic fusion-related assays targeted single oncogenic fusions, such as the detection of BCR-ABL1 fusions to monitor chronic myeloid leukemia (CML) methods; such assays were effective because BCR fuses exclusively with ABL1. By contrast, KMT2A fusions are challenging to target because KMT2A is known to fuse with up to 80 binding partners.

In some aspects, the disclosed assay is configured to detect KMT2A fusions involving multiple oncogenic fusion partners. In various aspects, the disclosed assay may be configured to detect a plurality of different KMT2A fusions without limitation. By way of non-limiting example, Table 1 summarizes a plurality of oncogenic KMT2A fusions associated with AML/BALL leukemia types that are detectable using the methods of the disclosed assay. In one embodiment, the disclosed assay is configured to detect multiple KMT2A fusion partners to cover 80% of the most common KMT2A fusions seen in t-AML, including KMT2A-AF9 (MLLT1), KMT2A-AF4 (AFF1), KMT2A-AF6 (AFDN), KMT2A-ENL (MLLT1), and KMT2A-ELL (ELL) fusions, wherein the partner gens is shown in parentheses. The disclosed assay provides a novel mechanism to target and detect these fusions for residual disease detection with high sensitivity.

TABLE 1
Oncogenic KMT2A Fusions

| Tumor Type | Fusion | Fusion |
|---|---|---|
| Leukemia | KMT2A_ABI1 | KMT2A_MLLT1 |
| Leukemia | KMT2A_ACTN4 | KMT2A_MLLT1 |
| Leukemia | KMT2A_AFDN | KMT2A_MLLT10 |
| Leukemia | KMT2A_AFF1 | KMT2A_MLLT11 |
| Leukemia | KMT2A_AFF3 | KMT2A_MLLT3 |
| Leukemia | KMT2A_AFF4 | KMT2A_MLLT6 |
| Leukemia | KMT2A_ARFIP1 | KMT2A_MYO1F |
| Leukemia | KMT2A_ARHGEF37 | KMT2A_MYOCD |
| Leukemia | KMT2A_CREBBP | KMT2A_PICALM |
| Leukemia | KMT2A_DCP1A | KMT2A_RELA |
| Leukemia | KMT2A_ELL | KMT2A_SARNP |
| Leukemia | KMT2A_EP300 | KMT2A_SEPTIN2 |
| Leukemia | KMT2A_FNBP1 | KMT2A_SEPTIN6 |
| Leukemia | KMT2A_LASP1 | KMT2A_SEPTIN9 |
| Leukemia | KMT2A_LYN | KMT2A_USP2 |
| Leukemia | KMT2A_MAML2 | KMT2A_USP9X |
| Leukemia | KMT2A_MATR3 | |

As described in the examples, the method for KMT2A fusion detection by dual-color ddPCR in one embodiment achieved sensitive detection over several logs of dynamic range and reliably excluded patient samples and cell lines that did not harbor KMT2A fusions. The disclosed assay improved upon qPCR strategies due to its ease of use, accurate transcript quantification, ease of multiplex analysis, and flexibility to modify or expand the target panel and reproducibility. The disclosed assay does not require standard curves for calibration, as the quantification is absolute. Prior efforts to develop a digital quantification for KMT2A fusions have been limited due to the promiscuous nature of KMT2A fusions that include over 80 known KMT2A fusion partners to date.

In some aspects, the disclosed assay may be configured to target the five most common KMT2A fusion partners that encompass approximately 80% of KMT2A rearranged AML cases. As demonstrated in the Examples, the limit of detection for the disclosed assay was variable based on the amount of input material, but in the dilution experiments described herein, cells harboring KMT2A fusions were reliably identified when their estimated abundance was 10 cells out of 2 million total cells.

In various aspects, the disclosed assay is configured to detect at least one or more possible oncogenic fusions without limitation. In some aspects, the disclosed assay is configured to detect one oncogenic fusion. In these aspects, multiple assays, each configured to detect a different oncogenic fusion associated with a cancer type, may be used to detect at least a subset of the total oncogenic fusions associated with the cancer type. In other aspects, the disclosed assay is a pooled assay configured to detect two or more oncogenic fusions associated with a cancer type simultaneously.

Without being limited to any particular theory, the sensitivity of the assay is enhanced by the inclusion of higher numbers of possible oncogenic fusions detected. By way of non-limiting example, the promiscuity of KMT2A to translocate with at least 80 known fusion partners has previously hindered the development of any sensitive RT-qPCR or ddPCR assays to detect KMT2A fusions associated with t-AML. In one aspect, the disclosed assay targets a subset of 80 possible KMT2A fusions associated with t-AML. In various aspects, the subset of oncogenic fusions is selected to account for a percentage of the total known oncogenic fusions including, but not limited to, at least 50%, at least 55%, at least 60%, at least 65%, at least 70%, at least 75%, at least 80%, at least 85%, at least 90%, at least 95%, at least 98%, or at least 99% of the total known oncogenic fusions associated with t-AML or other cancer. In one non-limiting example, the disclosed assay targets five oncogenic KMT2A fusions representing at least 80% of the total oncogenic KMT2A fusions known to be associated with t-AML. The disclosed assay can sensitively and specifically detect KMT2A fusions by ddPCR, even if limited to the most common KMT2A fusions. Given the ease of assay development and validation, additional KMT2A fusions could be added to the assay with minimal cost and effort.

In various aspects, the disclosed assay makes use of primer/probe pairs targeting portions of DNA or RNA sequences positioned upstream and downstream of the oncogenic fusion to be detected using droplet digital PCR (ddPCR) methods. Any suitable ddPCR system may be used for the disclosed assay including, but not limited to, a QX200 Droplet Digital PCR System (Bio-Rad). The DNA or RNA sequences bridging the oncogenic fusion may be obtained from any suitable source including, but not limited to, published sequences. In some aspects, RNA samples are expressed into complementary DNA (cDNA) before ddPCR.

Each primer/probe pair includes a primer targeting a portion of a cDNA sequence and a probe configured to anneal within the corresponding primer. In various aspects, an upstream primer/probe pair targeting a portion of the cDNA sequence of the oncogenic fusion upstream of a break and a downstream primer/probe pair targeting a portion of the cDNA sequence of the oncogenic fusion downstream of the break within the fusion partner region are used. In various aspects, the upstream probe and downstream probe are labeled with different reporters to facilitate the detection of the oncogenic fusion.

In one exemplary embodiment, the disclosed assay makes use of two-color ddPCR detection that makes use of an upstream probe labeled with a first fluorescent reporter and a downstream probe labeled with a second fluorescent reporter. In various aspects, the first and second fluorescent reporters are independently selected from fluorescein and hexachlorofluorescein

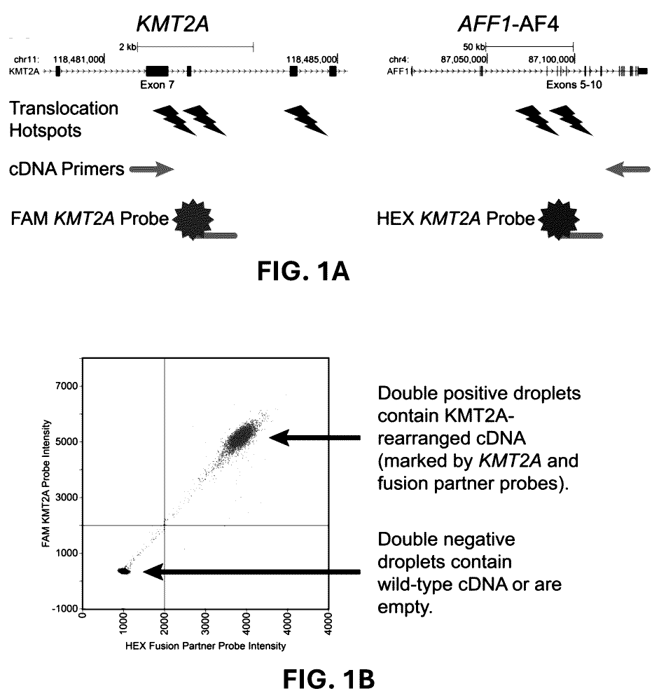

FIG. 1A illustrates an overall design approach to the methods of oncogenic fusion detection described herein as applied to the ddPCR detection and quantification of a KMT2A-AF4 fusion. As illustrated in FIG. 1A, forward and reverse cDNA primers are provided to produce cDNA from an RNA sample containing the oncogenic fusion. As shown in FIG. 1A, a forward PCR primer targeting a KMT2A start codon of the KMT2A fragment upstream of the fusion site and at least one reverse PCR primer targeting a start codon of the partner gene (AF4) downstream of the fusion site are used.

Referring again to FIG. 1A, the upstream probe is configured to configured to anneal to the KMT2A fragment between the KMT2A start codon and the fusion site and the downstream probe is configured to anneal between the partner (AF4) start codon and the fusion site. As illustrated in FIG. 1A, the first fluorescent reporter of the upstream probe is fluorescein (FAM), and the second fluorescent reporter of the downstream probe is hexachlorofluorescein (HEX).

FIG. 1B is a representative output of the ddPCR analysis using the dual-color probes as described above, Each droplet is classified as double positive droplets corresponding to droplets containing signals from both the first and second fluorescent reporters, and double negative droplets corresponding to either empty droplets or droplets containing wild-type (i.e. non-fused) KMT2A.

In various aspects, the disclosed assay may be implemented to detect and quantify single types of oncogenic KMT2A fusions as described above. In some aspects, multiple assays may be used to detect and quantify multiple types of oncogenic KMT2A fusions individually to quantify the types and abundances of individual oncogenic KMT2A fusions. For assays configured to detect a single type of oncogenic KMT2A fusions, double positive ddPCR results (see FIG. 1B) indicate the presence and abundance of the single type of oncogenic KMT2A fusion. Double negative ddPCR results are associated with empty cells, cells containing wild-type KMT2A, and/or cells containing oncogenic KMT2A fusion types other than the single fusion types for which the assay was designed.

In various other aspects, the disclosed assay may be implemented to identify two or more types of oncogenic KMT2A fusions using pooled primer-probe pairs. In this aspect, the assay includes at least one upstream PCR primer targeting a KMT2A start codon of the KMT2A fragment upstream of the fusion site and multiple reverse PCR primers; each reverse PCR primer targets a start codon of each partner gene downstream of the fusion site. In various aspects, each reverse primer corresponds to a different partner gene of a single oncogenic KMT2A fusion type. Additionally in this aspect, the assay includes the at least one first probe as described as above and multiple second probes. Each second probe is configured to anneal to the partner gene fragment of a single fusion type between the partner gene start codon and the fusion site. The PCR of the different fusion types is achieved using each reverse primer targeting each partner protein for each fusion type and the forward primer that is shared for the PCR of all fusion types, since all the fusion types share the upstream KMT2A in common.

In various aspects, the pooled assays using multiple downstream primers and probes targeting different partner genes associated with different types of oncogenic KMT2A fusions, the ddPCR output appears similar to the representative output illustrated in FIG. 1B. For the pooled assay, because all downstream probes targeting different partner proteins share the same type/color of fluorescent reporter, each double positive result corresponds to any of the droplets containing any of the types of KMT2A fusions for which the assay is targeted. Consequently, the abundance of KMT2A fusion expression derived from the ddPCR results represent a pooled total of all types of KMT2A fusions for which the assay is targeted. For this pooled-type assay, double-negative results represent empty cells, cells containing wild-type KMT2A, and/or cells containing types of KMT2A fusions different from the fusion types targeted by the assay's primer-probe pairs.

In various aspects, the design and methodology of the disclosed assay render the disclosed assay readily adjustable to include or remove the detection of a particular type of KMT2A fusions by including or removing primer-probe pairs targeting the partner gene of a particular KMT2A fusion type. By way of one non-limiting example, the assay may contain primer-probe pairs targeting a group of KMT2A fusion types representing 80% of all KMT2A fusions observed in AML patients, as described in further detail in the examples below. By way of another non-limiting example, the disclosed assay may be tailored to include patient-specific types of some aspect, an assay may be tailored to an individual patient's types of KMT2A fusions by including primer-probe pairs targeting the partner genes of fusion types identified by genomic sequencing of the patient's leukemia or other cancer.

In various aspects, the forward and reverse PCR primers are configured to produce an amplicon that includes the KMT2A fragment and the partner gene fragment flanking the fusion region. In various aspects, the forward and reverse PCT primers are configured to produce an amplicon with a length ranging from about 300 base pairs (bp) to about 500 bp. In various other aspects, the forward and reverse PCT primers are configured to produce an amplicon with a length ranging from about 300 bp to about 320 bp, from about 310 bp to about 330 bp, from about 320 bp to about 340 bp, from about 330 bp to about 350 bp, from about 340 bp to about 360 bp, from about 350 bp to about 370 bp, from about 350 bp to about 380 bp, from about 370 bp to about 390 bp, from about 380 bp to about 400 bp, from about 390 bp to about 410 bp, from about 400 bp to about 420 bp, from about 410 bp to about 430 bp, from about 420 bp to about 440 bp, from about 430 bp to about 450 bp, from about 440 bp to about 460 bp, from about 450 bp to about 470 bp, from about 460 bp to about 480 bp, from about 470 bp to about 490 bp, or from about 480 bp to about 500 bp.

In various other aspects, the amplicons and probes are configured to provide favorable annealing conditions comprising an annealing temperature Tₘ of about 60° C. In various other aspects, the amplicons and probes are configured to provide favorable annealing conditions comprising an annealing temperature Tₘ of about 52° C., of about 53° C., of about 54° C., of about 55° C., of about 56° C., of about 57° C., of about 58° C., of about 59° C., of about 60° C., of about 61° C., of about 62° C., of about 63° C., or of about 64° C.

Although the oncogenic fusion detection methods as described herein are described in terms of the analysis of RNA expression abundances, the disclosed detections may be modified to analyze DNA expression patterns indicative of oncogenic fusions.

In some aspects, the disclosed assay detects oncogenic fusions by detecting RNA within a biological sample. Although RNA is typically less stable than DNA and requires fusion expression for detection, RNA is well-suited for detecting oncogenic fusions within genes characterized by a heterogeneity of translocation breakpoints. By way of non-limiting example, the KMT2A gene is characterized by heterogeneity of translocation breakpoints occurring predominantly between exons 7 and 11. Without being limited to any particular theory, an assay that detected oncogenic fusions using DNA-based measurements would require considerably more primer/probe pairs to cover the same set of translocation breakpoints as a corresponding RNA-based assay.

In some aspects, the disclosed RNA-based assay may incorporate normalization of the measured fusion transcript abundance to housekeeping genes. Without being limited to any particular theory, the RNA-based assay relies on the expression of the oncogene for detection of the RNA indicative of oncogenic fusions, and consequently, transcript abundance does not necessarily correlate with leukemic or other cancer burden. In one embodiment of the disclosed assay, KMT2A fusion abundance is compared to wild-type KMT2A expression, which should have similar gene regulation and expression levels. As illustrated in the Examples herein, this normalization scheme was validated by cell line dilution experiments that used the ratio of KMT2A fusions to KMT2A wild-type transcript abundance to estimate the fraction of cells harboring KMT2A fusions; the estimated fraction of cells matched abundance at each serial dilution. Without being limited to any particular theory, similar normalization of fusion transcript abundance to housekeeping genes is employed in other ddPCR fusion assays.

In various aspects, the disclosed assay is suitable for use in the detection of residual disease after treatment. In one aspect, the disclosed assay is suitable for use in detecting AML harboring KMT2A fusions and for early KMT2A fusion detection in individuals at risk for developing KMT2A fusion-driven t-AML. For AML patients with KMT2A fusion-driven disease, this assay could augment or replace standard methods for residual disease detection during treatment, after hematopoietic stem cell transplant, or during long-term surveillance. This would be similar to the dramatic improvements in CML treatment decision-making made possible by sensitive qPCR-based BCR-ABL1 fusion detection. In one aspect, the disclosed assay may be compared directly to MPFC and morphology data for residual disease assessment during the treatment of AML.

In various aspects, the disclosed assay may be used classify a cancer patient as having measurable residual disease based on the ddPCR measurement of abundance of expression of the oncogenic fusions. In some aspects, the cancer patient is classified as having measurable residual disease if the abundance of expression of at least one oncogenic KMT2A fusion falls above a threshold value of 0.001% as measured by ddPCR for the oncogenic KMT2A fusion or fusions. In various other aspects, the threshold value may be 0.01%, 0.001%. 0.0001%, or 0.00001% as measured by ddPCR for the oncogenic KMT2A fusion or fusions.

In some aspects, the classification of the patient may be used to inform treatment decisions by a practitioner. By way of non-limiting example, a practitioner may recommend administering additional treatment to a cancer patient if the patient is classified as having measurable residual disease, or the practitioner may recommend terminating treatment and/or periodic monitoring if the patient is not classified as having measurable residual disease.

In some aspects, the disclosed assay may be used to monitor a post-treatment or otherwise at-risk patient for incipient relapse, recurrence, or de novo development of a cancer. By way of non-limiting example, disclosed assay may be used as described above to evaluate whether the patient is classified as having measurable residual disease and therefore in need of a treatment.

In some aspects, the disclosed assay may be incorporated into interventional clinical trials and the detected oncogenic fusions may be used as a biomarker for treatment decision-making. In other aspects, the disclosed assay may be used to identify pre-malignant KMT2A fusions in patients receiving TOP2 inhibitors and at risk for t-AML. Without being limited to any particular theory, t-AML risk is thought to be highest for several cancers affecting children and adolescents including Ewing's sarcoma, Hodgkin lymphoma, and neuroblastoma. While rare, t-AML is extremely difficult to treat in a population that has already received large lifetime doses of chemotherapy. In other additional aspects, the disclosed assay may be used to identify at-risk individuals and to identify a need for intervention before fulminant disease develops.

Although the methods of detecting oncogenic fusions disclosed herein analyze RNA or DNA expression of cells isolated from blood samples or bone marrow samples, in various aspects the method may be modified to analyze RNA or DNA from cancer cells isolated from other samples including, but not limited to solid tumor samples, cell-free DNA from blood samples, and any other suitable sample containing cancer cells.

In various aspects, the disclosed assay may be modified as described herein to detect any oncogenic fusion associated with any type of cancer without limitation. Non-limiting examples of oncogenic fusions suitable for detection using the methods disclosed herein are summarized in Table 1 below.

TABLE 1
Exemplary Oncogenic Fusions

| Tumor Type | Fusion | |
|---|---|---|
| Brainstem glioma- | FGFR1_TACC1 | MTMR2_MAML2 |
| Diffuse intrinsic | FGFR3_TACC3 | TSPAN12_MET |
| pontine glioma | HNRNPA2B1_MET | |
| Brain Tumor | ALK_BIRC6 | GKAP1_NTRK2 |
| | ATXN1_NUTM2B | GOPC_ROS1 |
| | BEND5_NTRK2 | HIP1_ALK |
| | BTBD1_NTRK3 | HIP1_MET |
| | C11orf95_RELA | KIAA1549_BRAF |
| | CIC_NUTM1 | KLHL7_MET |
| | CLIP2_ALK | MN1_CXXC5 |
| | EEF1G_ROS1 | MN1_PATZ1 |
| | EGFR_ZNRF3 | MYB_QKI |
| | EML1_NTRK2 | MYBL1_MAML2 |
| | EP300_BCOR | MYBL1_QKI |
| | ETV6_NTRK3 | PPP1CB_ALK |
| | EWSR1_CREM | SPTBN1_ALK |
| | FGFR1_TACC1 | TPM3_NTRK1 |
| | FGFR2_CTNNA3 | VCL_NTRK2 |
| | FGFR2_INA | |
| Cavernoma | KIAA1549_BRAF | |
| Dysembryoplastic | FGFR1_TACC1 | PDGFB_LRP1 |
| neuroepithelial | FGFR2_INA | |
| tumor (DNET) | | |
| Brain Tumor (EPD) | C11orf95_MAML2 | MN1_PATZ1 |
| | C11orf95_NCOA2 | QKI_ARID1B |
| | C11orf95_RELA | YAP1_KDM2B |
| | C11orf95_YAP1 | YAP1_MAML2 |
| | EP300_BCOR | YAP1_MAMLD1 |
| Ewing's Sarcoma | EWSR1_FLI1 | |
| Ganglioglioma | FGFR2_SHTN1 | PPP1CB_ALK |
| | KCTD8_NTRK2 | TNS3_BRAF |
| | KIAA1549_BRAF | |
| Glial-neuronal tumor | GTF2I_BRAF | KIAA1549_BRAF |
| HGG | CCDC88A_ALK | PTPRZ1_MET |
| | CHD7_MYBL1 | TPM3_NTRK1 |
| | CLIP1_ROS1 | TRIM24_BRAF |
| | MN1_PATZ1 | ZCCHC8_ROS1 |
| LGG | AFAP1_NTRK2 | KIAA1549_BRAF |
| | AKAP9_BRAF | MACF1_BRAF |
| | ANTXR1_BRAF | MN1_PATZ1 |
| | BCAS1_RAF1 | MYB_QKI |
| | C11orf95_RELA | MYO5A_NTRK3 |
| | DCTN1_ALK | NAV1_NTRK2 |
| | DIAPH2_NTRK2 | PID1_BRAF |
| | ERC2_RAF1 | PID1_RAF1 |
| | ETV6_NTRK3 | PPP1CB_ALK |
| | FAM131B_BRAF | RNF130_BRAF |
| | FGFR1_TACC1 | SLMAP_NTRK2 |
| | FGFR3_TACC3 | TAX1BP1_BRAF |
| | FYCO1_RAF1 | TRIM24_ROS1 |
| | KANK1_NTRK2 | YAP1_FAM118B |
| MB | HMGA2_NCOR2 | PAX3_NCOA2 |
| | MACF1_SLC6A18 | |
| Meningioma | YAP1_FAM118B | YAP1_MAML2 |
| Metastatic secondary | EWSR1_FLI1 | TFG_ROS1 |
| tumors | | |
| Neurocytoma | FGFR1_TACC1 | |
| Neurofibroma Plexiform | ARHGEF2_NTRK1 | |
| Brain Tumor (not | EWSR1_FLI1 | KIAA1549_BRAF |
| reported) | | |
| Brain Tumor (other) | EWSR1_ATF1 | GOPC_ROS1 |
| Brain Tumor | FGFR2_CTNNA3 | NRCAM_PRKCB |
| Rhabdomyosarcoma | PAX3_NCOA1 | |
| Sarcoma | HEY1_NCOA2 | MBNL1_BRAF |
| | KMT2A_MLLT10 | |
| Supratentorial or | C11orf95_RELA | EWSR1_FLI1 |
| Spinal Cord PNET | | |
| Leukemia (AML) | BCR_ABL1 | NPM1_RARA |
| | CBFA2T3_GLIS2 | NUP214_ABL1 |
| | CBFA2T3_GLIS3 | NUP98_BPTF |
| | CBFB_MYH11 | NUP98_BRWD3 |
| | DDX3X_MLLT10 | NUP98_CADM1 |
| | DDX3Y_MLLT10 | NUP98_DDX10 |
| | DEK_NUP214 | NUP98_JADE2 |
| | EP300_ETV6 | NUP98_KDM5A |
| | ETV6_ABL1 | NUP98_NSD1 |
| | ETV6_FOXO1 | NUP98_PHF23 |
| | ETV6_INO80D | NUP98_PRRX1 |
| | EWSR1_ELF5 | NUP98_RAP1GDS1 |
| | EWSR1_ERG | NUP98_ZFX |
| | EWSR1_FEV | PICALM_AFF2 |
| | FUS_ERG | PICALM_MLLT10 |
| | FUS_FEV | PML_RARA |
| | FUS_FLI1 | PSIP1_NUP214 |
| | GATA2_ERG | PSPC1_ZFP36L1 |
| | HNRNPH1_ERG | RANBP2_ALK |
| | HSPA8_FLI1 | RBM15_MRTFA |
| | KAT6A_CREBBP | RUNX1_CBFA2T2 |
| | KAT6A_EP300 | RUNX1_CBFA2T3 |
| | KAT6A_NCOA2 | RUNX1_RUNX1T1 |
| | MAP2K2_DIS3L | RUNX1_USP42 |
| | MAP2K2_MLLT10 | RUNX1_ZFPM2 |
| | MLLT10_DDX10 | SET_NUP214 |
| | MN1_ETV6 | SFPQ_AGO1 |
| | MN1_FLI1 | SFPQ_ZFP36L2 |
| | MYB_CLINT1 | SNRNP70_FGFR1 |
| | MYB_GATA1 | SPTBN1_ALK |
| | NAP1L1_MLLT10 | STAG2_AFF2 |
| | NIPBL_ETV6 | TBL1XR1_RARB |
| | NONO_ZFP36L1 | TEC_MLLT10 |
| | NPM1_CCDC28A | XPO1_MLLT10 |
| | NPM1_HAUS1 | XPO1_TNRC18 |
| | NPM1_MLF1 | PAX5_DACH1 |
| Leukemia (BALL) | ACIN1_NUTM1 | PAX5_ESRRA |
| | ARID1B_ZNF384 | PAX5_ETV6 |
| | ATF7IP_JAK2 | PAX5_FAM184B |
| | BCR_ABL1 | PAX5_FBRSL1 |
| | BCR_JAK2 | PAX5_FOXP1 |
| | BICD2_JAK2 | PAX5_GREB1L |
| | BRD9_NUTM1 | PAX5_JAK2 |
| | EBF1_PDGFRB | PAX5_NCOA5 |
| | EP300_ZNF384 | PAX5_NOL4L |
| | ETV6_ABL1 | PAX5_ZNF276 |
| | ETV6_FAM169B | PICALM_MLLT10 |
| | ETV6_JAK2 | PPFIBP1_JAK2 |
| | ETV6_NTRK3 | RCSD1_ABL1 |
| | ETV6_RUNX1 | RCSD1_ABL2 |
| | ETV6_SKAP1 | SMARCA2_ZNF362 |
| | GOLGB1_JAK2 | SMARCA2_ZNF384 |
| | IKZF1_ZEB2 | SSBP2_CSF1R |
| | KLF3_PAX5 | SSBP2_FLT3 |
| | MEF2D_BCL9 | SSBP2_JAK2 |
| | MEF2D_DAZAP1 | SSBP2_PDGFRB |
| | MEF2D_HNRNPUL1 | STRN3_JAK2 |
| | MEF2D_KHDRBS1 | TAF15_ZNF384 |
| | MEIS1_FOXO1 | TCF3_FLI1 |
| | NUP214_ABL1 | TCF3_HLF |
| | PAG1_ABL2 | TCF3_PBX1 |
| | PAX5_AUTS2 | TCF3_ZNF384 |
| | PAX5_BCOR | TPR_JAK2 |
| | PAX5_CBFA2T3 | ZC3HAV1_ABL2 |
| Leukemia (TALL) | PICALM_MLLT10 | |
| Solid Tumor (EWS) | CIC_FOXO4 | EWSR1_FEV |
| | ETV6_NTRK3 | EWSR1_FLI1 |
| | EWSR1_ERG | FUS_NFATC2 |
| Solid Tumor (RHB) | PAX3_FOXO1 | PAX3_NCOA1 |
| | PAX3_INO80D | PAX7_FOXO1 |
| Solid Tumor (ST) | AGK_BRAF | LMNA_NTRK1 |
| | ASPSCR1_TFE3 | MAP3K8_GNG2 |
| | BCOR_CCNB3 | MAP3K8_STX7 |
| | CCDC6_RET | MAP3K8_SVIL |
| | CLIP1_ALK | MYB_NFIB |
| | COL1A1_PDGFB | NCOA4_RET |
| | DNAJB1_PRKACA | NPM1_ALK |
| | EML4_BRAF | NR1D1_MAML1 |
| | ETV6_NTRK3 | NRF1_BRAF |
| | EWSR1_ATF1 | PAX3_FOXO1 |
| | EWSR1_ERG | PDE4DIP_NTRK1 |
| | EWSR1_FLI1 | RAB10_ALK |
| | EWSR1_KLF15 | SQSTM1_ALK |
| | EWSR1_WT1 | SS18_SSX1 |
| | GSN_NTRK1 | TFG_ROS1 |
| | HEY1_NCOA2 | TPM3_ALK |
| | JAKMIP2_BRAF | TPM3_NTRK1 |
| | KIAA1217_RET | VCL_RET |
| | KMT2A_MAML2 | YWHAE_NUTM2B |

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| | Ile, Val, Met, Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, oncogenic fusions may be created in cell lines for use in the development and validation of the disclosed assays for detecting oncogenic fusions. In some aspects, oncogenic fusions may be created in cell lines using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N) 20NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome.

For example, the methods as described herein can comprise a method for altering a target polynucleotide sequence in a cell comprising contacting the polynucleotide sequence with a clustered regularly interspaced short palindromic repeats-associated (Cas) protein.

### Screening

In some aspects, the disclosed method of detecting oncogenic fusions may be used as part of various screening methods. In these aspects, changes in the expression of oncogenic fusions in cancer cells may be used to evaluate the efficacy of a candidate treatment composition or to characterize biochemical pathways modulated by a candidate treatment composition.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for use. Such kits can facilitate the performance of the methods for detecting oncogenic fusions described herein. When supplied as a kit, the different components of the composition can be packaged in separate containers and admixed immediately before use. Components include, but are not limited to, one or more upstream primer/probe pairs, one or more downstream primer/probe pairs, solubilizers, and sterile packaging. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Development and Validation of Droplet Digital PCR Assay for Oncogenic KMT2A Fusion Detection

To develop and validate a droplet digital PCR (ddPCR) assay to detect and quantify oncogenic KMT2A fusions, the following experiments were conducted.

There are at least 80 known KMT2A fusion partners, but about 80% of KMT2A fusions involve just five exome partners-AF9, AF6, AF4, ELL and ENL. Given the varied fusion partners, it is challenging to detect these fusions using qPCR technology.

Another droplet PCR technology, droplet digital PCR (ddPCR) technology was used to develop and assay that provided for the detection of the five most common KMT2A fusions accounting for the vast majority (ca. 80%) of all oncogenic KMT2A fusions found in therapy-related AML (t-AML) patients. The ddPCR assay was benchmarked using cell lines and primary patient samples with KMT2A fusions. The assay described below provided an inexpensive, rapid, sensitive, and specific platform for KMT2A fusion detection that potentially improves measurable residual disease (MRD) detection for AML patients with KMT2A fusions and enables screening for patients at risk for developing t-AML after receiving TOP2 inhibitor therapy.

### Materials and Methods

### Cell Lines

Human cell lines known to harbor KMT2A fusions were used to design, benchmark and validate the ddPCR assay. Cell lines used were THP-1 (KMT2A-AF9), MOLM-13 (KMT2A-AF9), MV4-11 (KMT2A-AF4), OCI-AML2 (KMT2A-AF6) and KOPN8 (KMT2A-ENL). A t-AML patient sample harboring the KMT2A-ELL fusion was used to design and test the KMT2A-ELL reagents. Cell lines without KMT2A fusions were used as controls including K562, HEL, Kasumi, Jurkat and OCI-AML3. For each ddPCR experiment, cell lines harboring a KMT2A fusion and cell lines without a KMT2A fusion were used as controls. THP-1 and HEL cells were grown in RPMI (ATCC 30-2001), 10% heat-inactivated fetal bovine serum (HI-FBS; Gibco), and 1% Penicillin-Streptomycin (P/S; Gibco) with 0.05 mM B-mercaptoethanol (Sigma-Aldrich) added to the THP-1 media. MOLM-13, Jurkat and KOPN8 cells were grown in RPMI (Gibco), 10% HI-FBS, and 1% P/S. Kasumi cells were grown in RPMI (Gibco), 20% HI-FBS and 1% P/S. The MV-4-11 cells were grown in IMDM (Gibco), 10% HI-FBS and 1% P/S. OCI-AML2 and OCI-AML3 cells were grown in Mem Alpha (Gibco), 20% HI-FBS and 1% P/S.

### Patient Samples

Cryopreserved human t-AML patient samples were banked at Washington University in St. Louis. All t-AML patients provided written informed consent for tissue repository and genomic sequencing in accordance with protocol #201011766 approved by the Washington University in St. Louis Institutional Review Board. De-identified control patient peripheral blood (PB) or bone marrow (BM) samples were obtained according to a protocol approved by the Washington University Human Studies Committee (WU 01-1014).

RNA Extraction and cDNA Synthesis

RNA was extracted from cell lines and patient samples using the RNeasy Plus Mini Kit (Qiagen). Up to 1×10⁶ cells were processed per experiment. RNA was extracted per manufacturer recommendations without modification. The RNAeasy spin columns were eluted with 25-50 uL RNase free water. The RNA concentration was quantified using Qubit Fluorometric Quantification (ThermoFisher Scientific). Complementary DNA (cDNA) was synthesized using Superscript IV VILO (ThermoFisher Scientific). Synthesized cDNA molecules were stored at −20° C.

### Droplet Digital PCR Primer/Probe Design

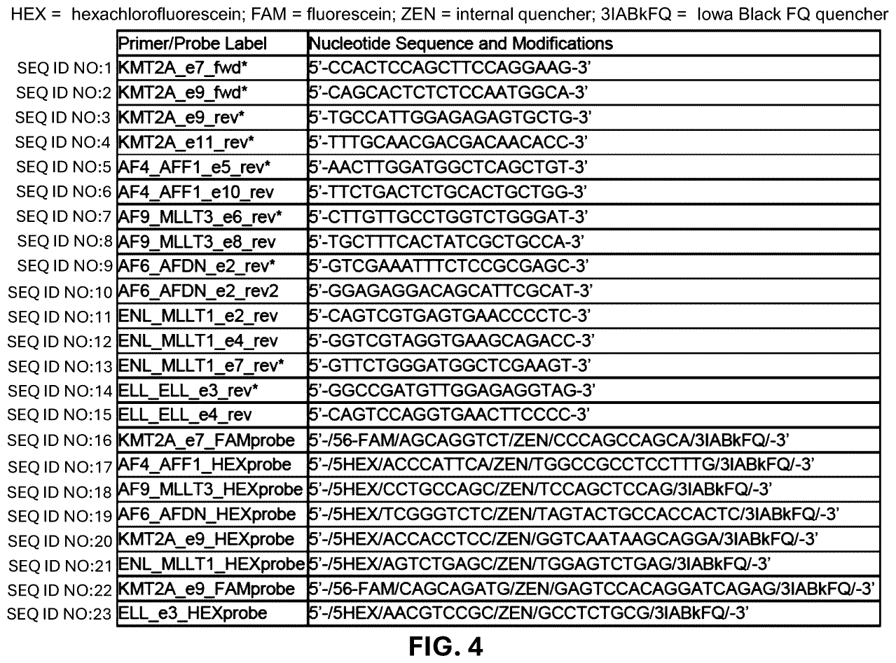

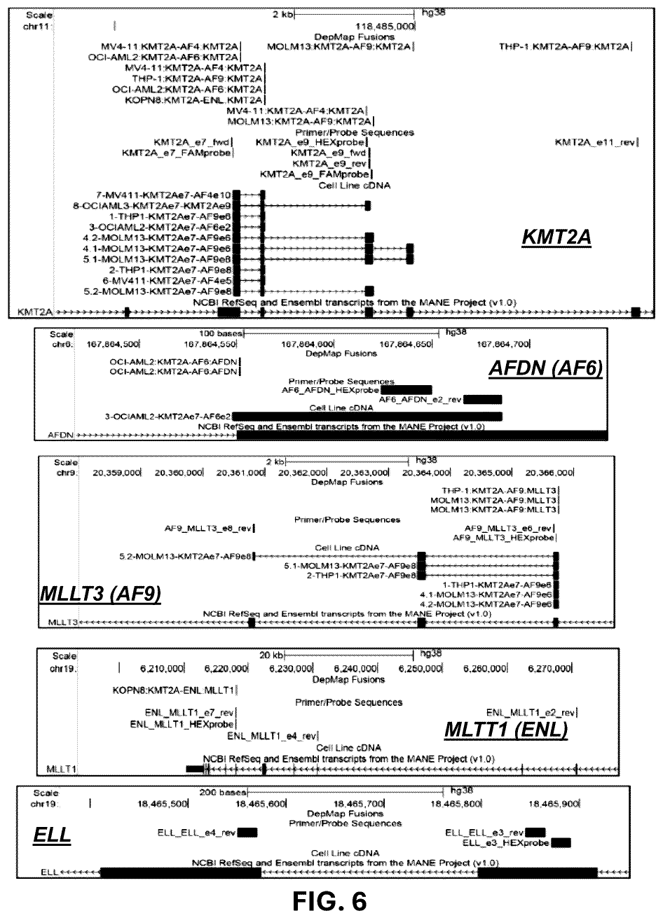

The genomic locations for the translocation in each cell line harboring a KMT2A fusion was identified from the Cancer Dependency Map (https://depmap.org/portal/; last accessed Dec. 17, 2021) and used to design primer sequences to span the fusions (FIG. 1A, FIG. 6, FIG. 4). Primers and probes were designed using Primer3Plus. Multiple exonic primers were designed for each translocation partner. Fusion-specific cDNA amplicons generated from cell lines harboring a KMT2A fusion were Sanger sequenced and mapped to the hg38 reference genome using BLAT to verify the fidelity of the primer pairs (FIG. 6). Prime Time fluorescent probes (Integrated DNA Technologies) were designed to anneal within the fusion-specific primers to add sensitivity and specificity to the assay (FIG. 1A, FIG. 6, FIG. 4). For each fusion, one fluorescein (FAM)-labeled probe was designed to anneal to KMT2A upstream of the fusion and one hexachlorofluorescein (HEX)-labeled probe was designed to anneal to the fusion partner downstream of the fusion. A control primer/probe pair was designed to tag the wild-type KMT2A cDNA with FAM and HEX labeled probes annealing in KMT2A exons.

### Droplet Digital PCR Reaction Conditions.

Droplet digital PCR experiments were conducted on the QX200 Droplet Digital PCR System (Bio-Rad). For each ddPCR reaction, the input cDNA was diluted to ensure that <330 ng cDNA was inputted per reaction. Each ddPCR reaction was comprised of 10 uL 2×ddPCR Supermix for Probes no dUTP, 1000 nM fusion-specific primers, 250 nM fusion-specific probes, cDNA (max 330 ng), RNase/DNase-free water to 20 uL total. Droplets were generated on the QX200 Droplet Generator (Bio-Rad) per manufacturer instructions. Droplet PCR amplification occurred using the following thermocycler conditions, 94° C. for 10 seconds; 40 cycles of 94° C. for 30 seconds, 60° C. for 1 minute; 98° C. for 10 minutes; 4° C. hold. Amplification was followed by imaging on the QX200 Droplet Reader (Bio-Rad) and analyzed using the QuantaSoft Analysis Pro software package (Bio-Rad). Multiple negative (cell-lines without known KMT2A fusions) and positive (cell-lines with known KMT2A fusions) controls were used per experiment. For each sample analyzed for KMT2A fusions, a separate aliquot was analyzed using primers and probes that targeted the wild-type KMT2A locus spanning exon 7-9 to provide an estimate of wild-type transcript abundance (FIG. 4).

### Pooled Assay Conditions.

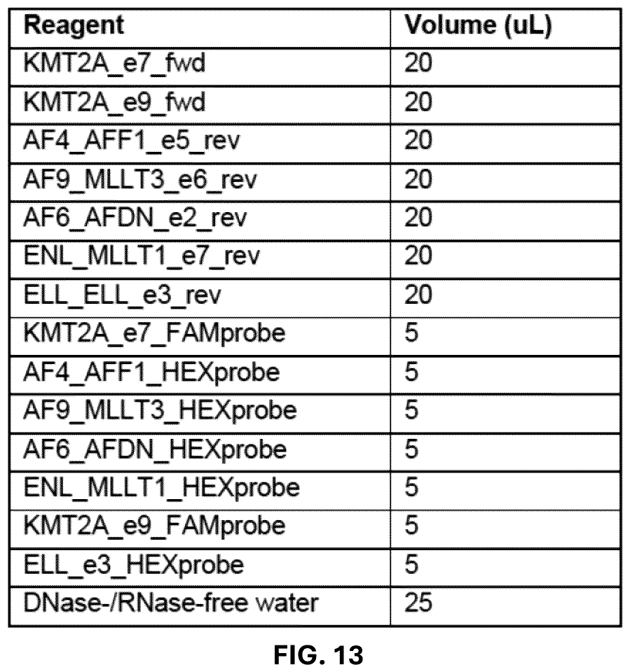

To enable simultaneous detection for all five KMT2A fusions targeted by the assay, a pooled ddPCR assay was designed and benchmarked. In a single reaction mixture, forward primers/probes for KMT2A exon 7 and KMT2A exon 9 were combined with reverse primers/probes for AF9 exon 6, AF4 exon 5, AF6 exon 2, ENL exon 7 and ELL exon 3 (FIG. 13). The pooled primer/probe mixture was 10× concentrated, such that 2 μL of the pooled primer/probe mixture added to a 20 uL ddPCR reaction would yield a final concentration of 1000 nM for each primer and 250 nM for each probe. Droplet generation, thermocycler conditions, droplet imaging and droplet analysis was performed as described above.

### Dilution Series Experiments

Cell lines harboring KMT2A fusions were serially diluted into OCI-AML3 cells (KMT2A wild-type). Cells were quantified by automated cell counter (Nexcelom). Beginning at a 50% mixture, 1,000,000 cells harboring a KMT2A fusion (e.g. THP-1) were mixed with 1,000,000 OCI-AML3 cells. From this mixture, 200,000 cells were removed and added to 1,800,000 OCI-AML3 cells to create the 5% mixture. The serial dilution was repeated to 0.0005%, at which point it was estimated there would be less than 10 cells harboring a KMT2A fusion remaining in the mixture. RNA was extracted and converted into cDNA as described above. The cDNA was diluted to 40 ng/uL and stored at −20° C. For the 50%, 5%, 0.5% and 0.05% dilutions, 80 ng of cDNA was used in a single ddPCR reaction well for fusion detection. For the 0.005% dilution 320 ng of cDNA was used per well for four ddPCR reaction wells (1280 ng total). For the 0.0005% dilution 320 ng of cDNA was used per well for eight ddPCR reaction wells (2560 ng total). Increasing the amount of cDNA included in the reaction was necessary at the 0.005% and 0.0005% dilutions to ensure that enough cells were assayed to enable detection of the rare KMT2A fusions. These serial dilution experiments simulated detecting rare leukemia cells harboring KMT2A fusions in patients and established the limit of detection for the assay.

### KMT2A Fusion Generation by Gene Editing

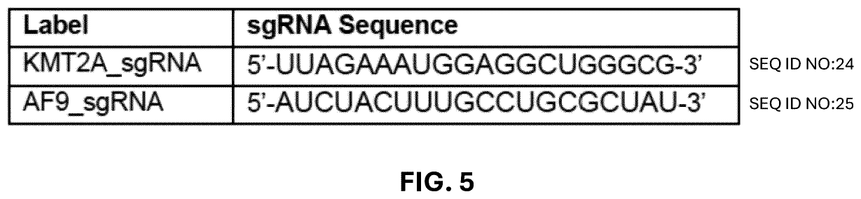

KMT2A fusions were generated using clustered regularly interspaced short palindromic repeats (CRISPR)/Cas9 gene editing. The fusion breakpoint occurred between exons 10 and 11 of KMT2A. Guide RNA (gRNA) sequences targeting the intronic regions of KMT2A and MLLT3/AF9 were synthesized (Synthego) using nucleotide sequences specified as SEQ ID NO:24 and SEQ ID NO:25 in the table of FIG. 5. Ribonucleoprotein (RNP) complexes were formed by incubating guide RNAs (120 μmol) with Cas9 (Integrated DNA Technologies) for 20 minutes at room temperature. Nucleofection was performed using the Neon Transfection System (ThermoFisher Scientific). The RNP complex was combined with 250,000 cells in 10 uL buffer R. Cells were electroporated using the settings 1700V, 20 ms, 1 pulse. After nucleofection, cells were cultured in appropriate media. From these cells, RNA was extracted using the above protocol and converted into cDNA. This cDNA was assayed using the KMT2A exon 9 and AF9 primer/probe combination to detect oncogenic fusions.

### Results

### Design of Droplet Digital PCR Assay

The standard dual color ddPCR assay for mutation detection utilizes FAM and HEX labeled probes overlapping a region of interest that differ by a single nucleotide or small indel. Competitive annealing of the probes at the locus provides the specificity to distinguish between wild-type and mutated DNA molecules. This standard method for variant detection is not compatible with fusion detection. To enable low frequency fusion detection, a novel cDNA-based dual color ddPCR assay in which each fusion was identified by PCR primers spanning the fusion was developed, and fluorescently labeled probes were nested flanking the fusion (FIG. 1, FIG. 4). In general, a FAM-labeled probe was designed to anneal within KMT2A upstream of the fusion and HEX-labeled probes were designed to anneal within the fusion partner downstream of the fusion.

### Droplet Digital PCR Benchmarking in Cell Lines

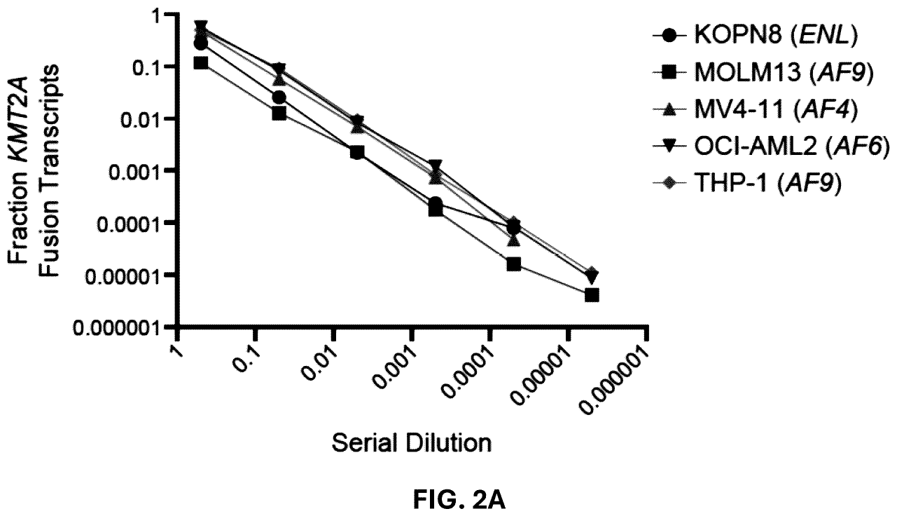

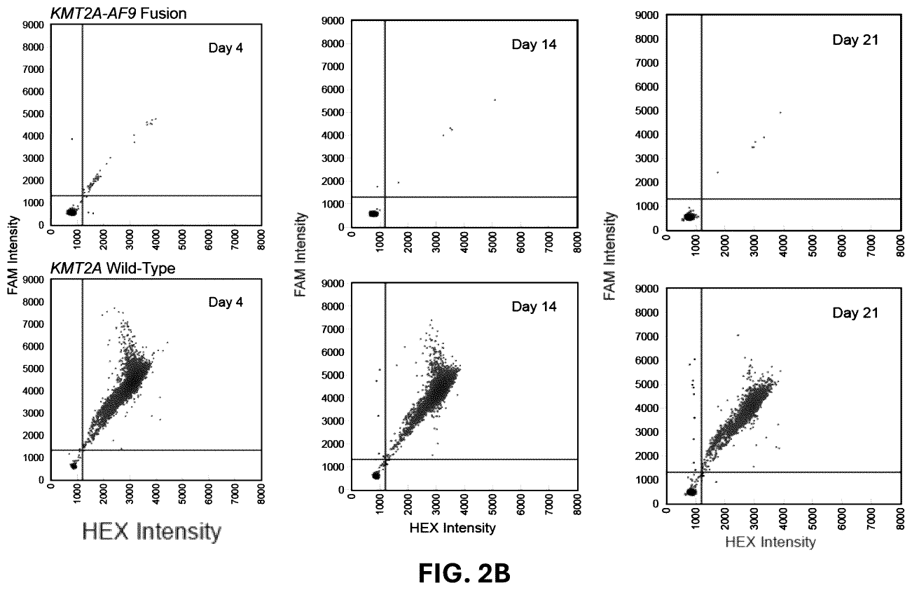

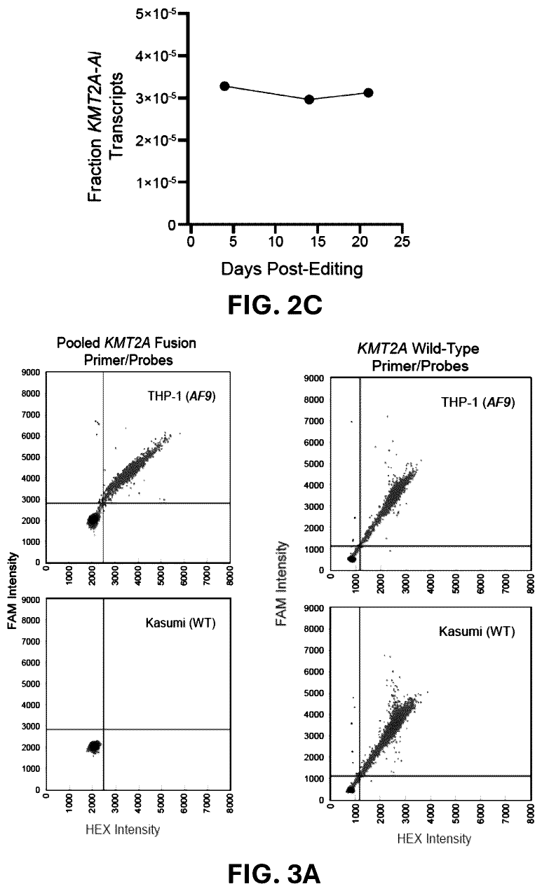

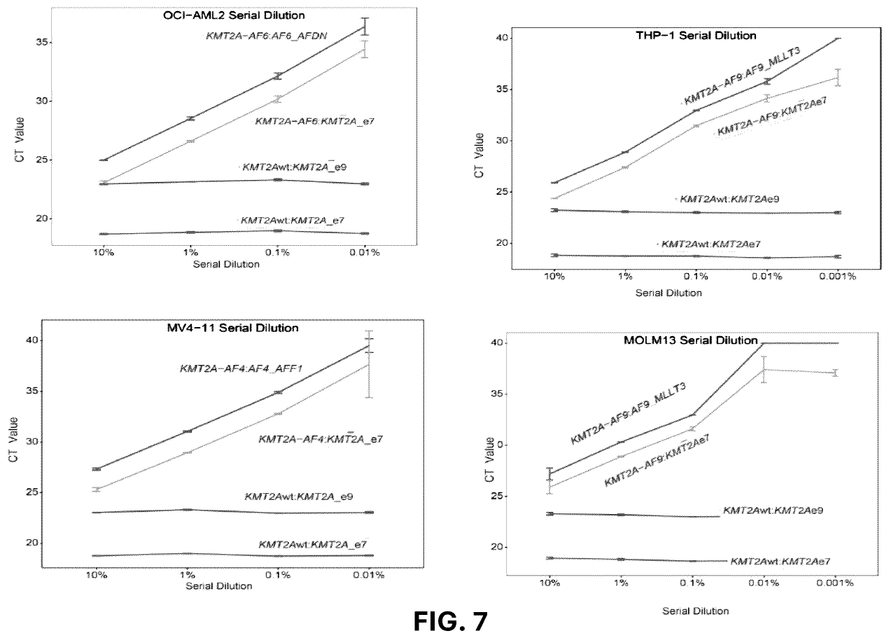

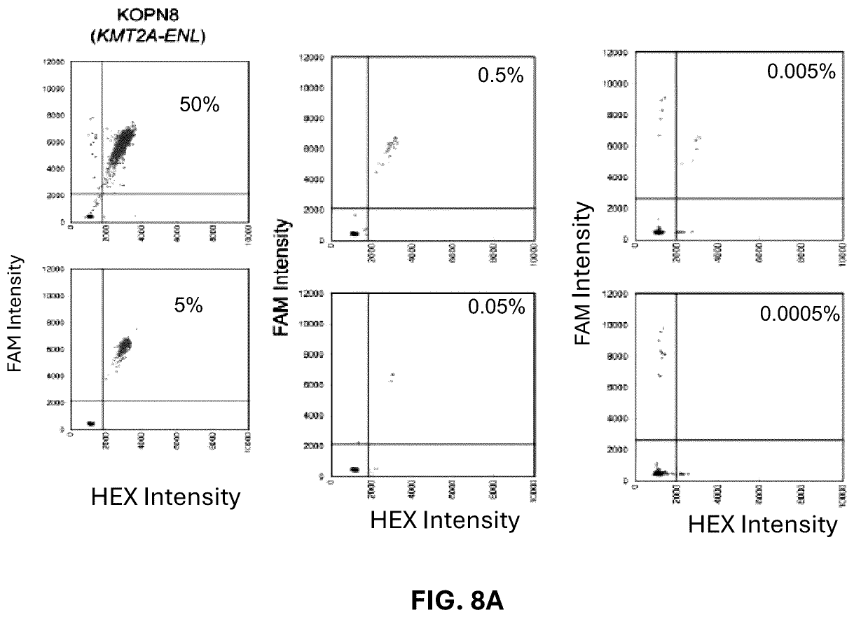

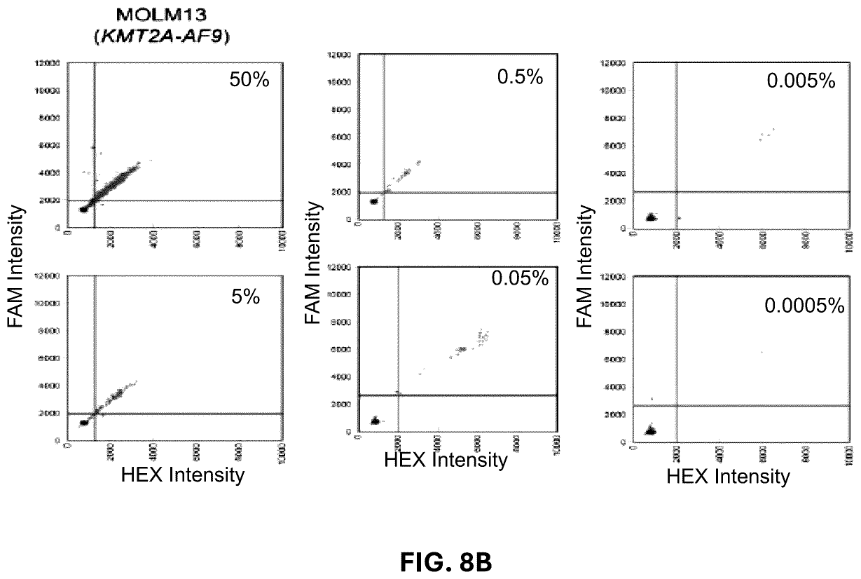

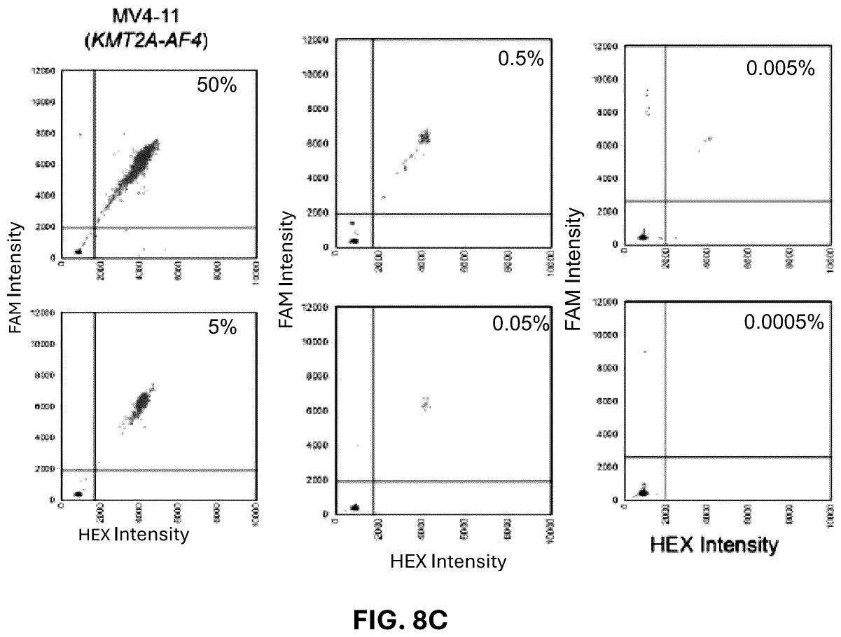

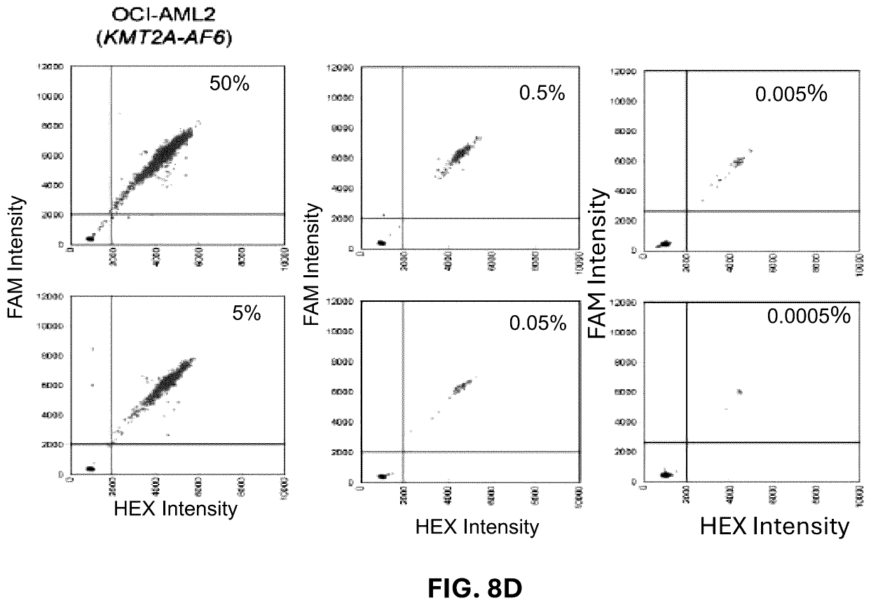

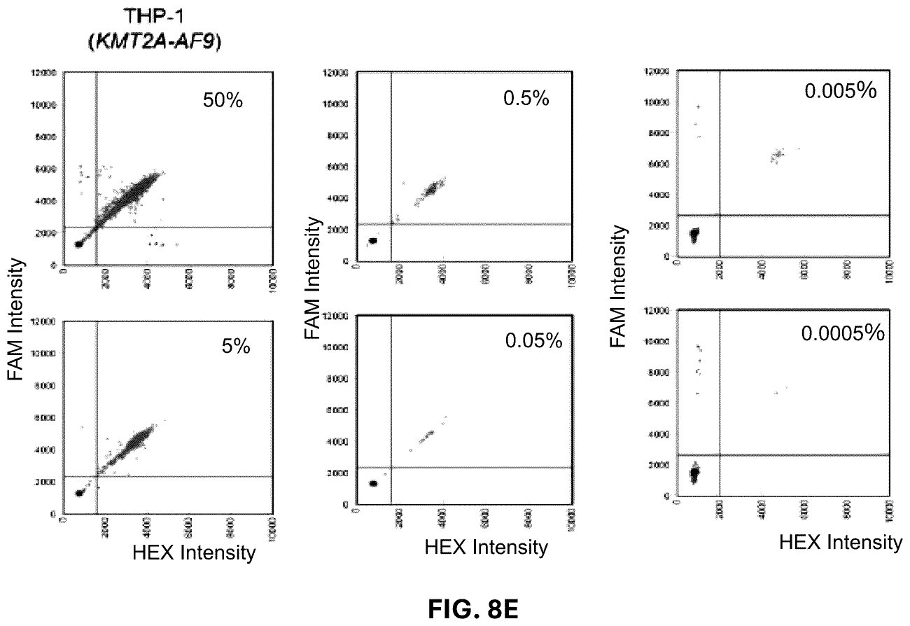

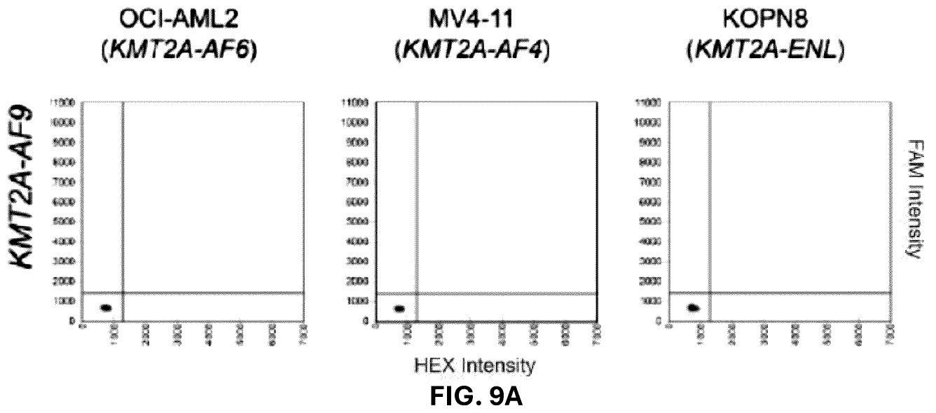

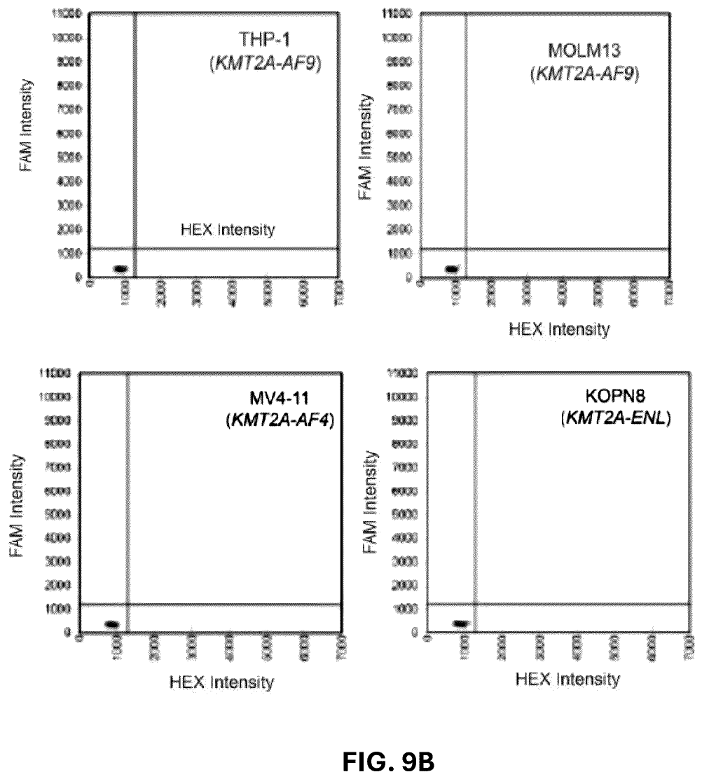

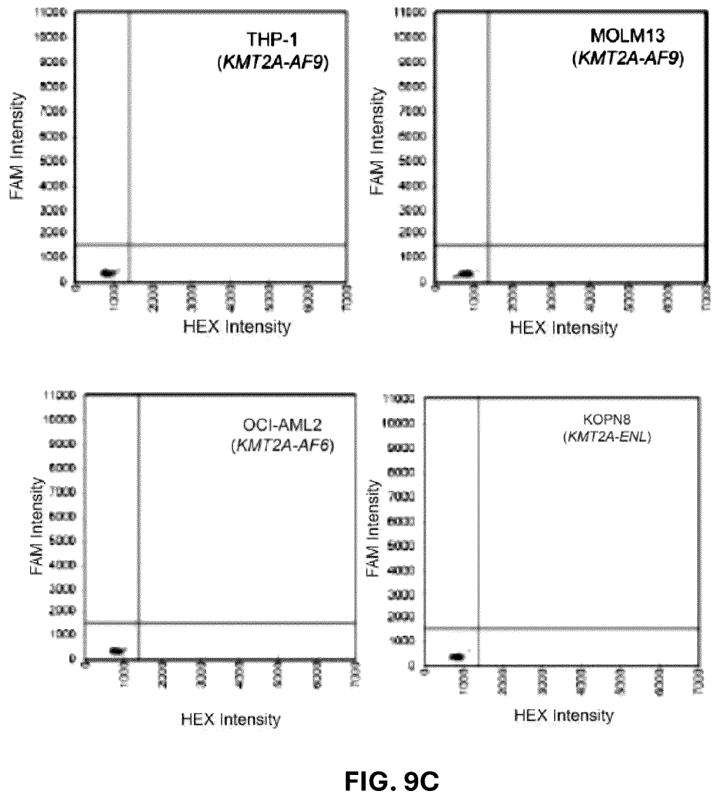

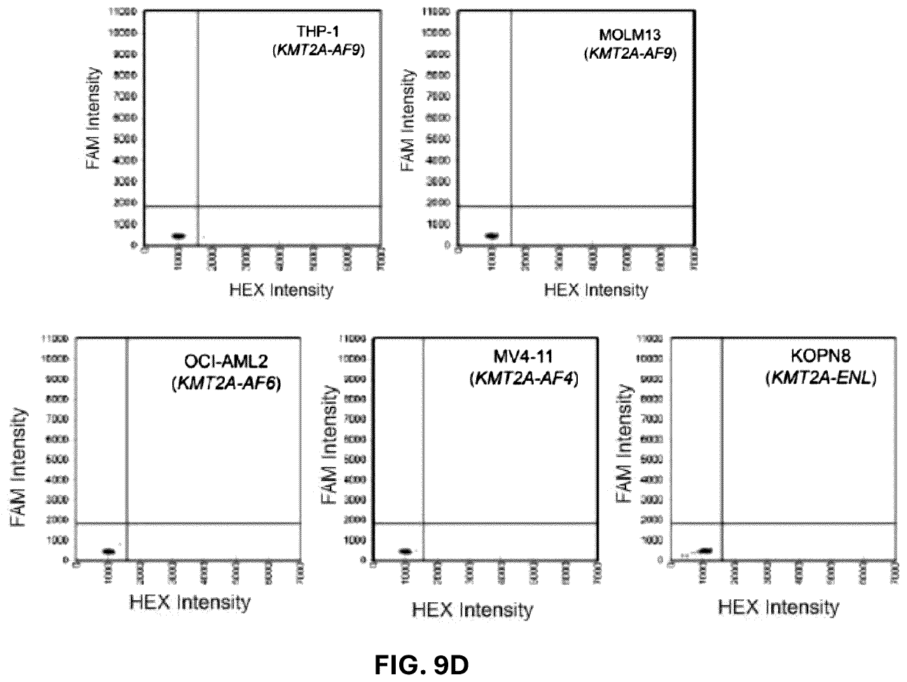

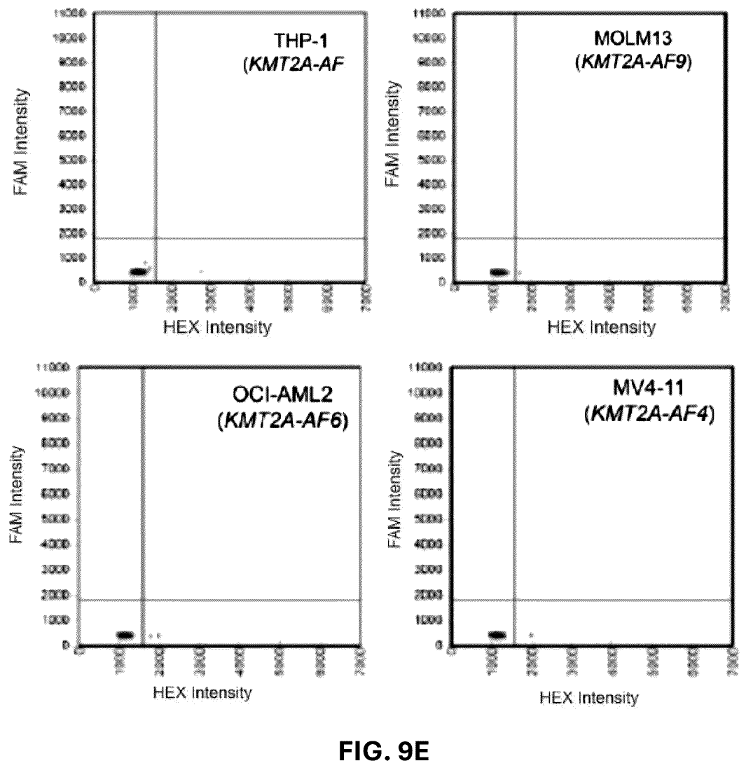

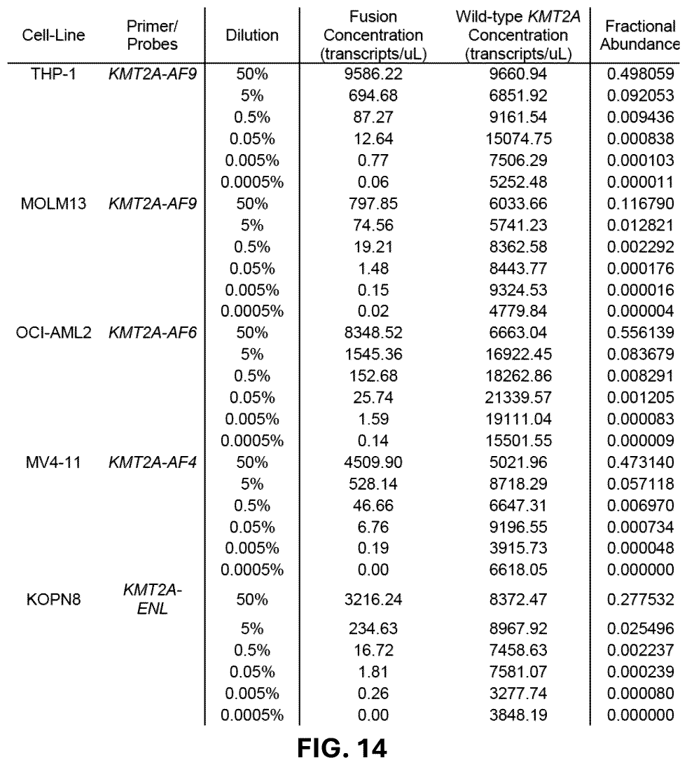

These primer/probe pairs were initially benchmarked in a dilution series experiment using bulk qPCR. Excellent performance of both KMT2A FAM-probe and fusion partner HEX-probes were observed across the panel (FIG. 7). For each qPCR experiment, a wild-type primer/probe pair was also incorporated in a separate reaction to estimate the abundance of the KMT2A wild-type transcript for comparison and ensure adequate sample preparation and loading. Once optimized, the primer/probe reagents were benchmarked on the ddPCR platform in a dilution series experiment (FIG. 8, FIG. 14). Cell lines harboring known KM T2A fusions were serially diluted into OCI-AML3 cells (KMT2A wild-type) and analyzed using ddPCR. The appropriate cell-type specific KMT2A fusion was detected over 5-6 logs of dynamic range in this experiment. The fractional abundance of KMT2A fusion transcripts was determined by calculating the concentration of KMT2A fusion transcripts and dividing by the total number of KMT2A (fusion and wild-type) transcripts detected and matched the expected KMT2A mutant cell line abundance over the entire dilution series (FIG. 2A). Primer/probe pairs designed to target a specific KMT2A fusion exhibited no off-target activity when assaying cell lines with different KMT2A fusions not targeted by the specific primer/pair (FIG. 9).

As proof-of-principle for KMT2A fusion detection in a setting that mimics patients receiving TOP2 inhibitor therapy, HEL cells with wild-type KMT2A were edited with CRISPR/Cas9 to introduce a KMT2A fusion. KMT2A fusions were detected four days after CRIPSR/Cas9 treatment and persisted at 14 and 21 days in culture (FIG. 2B). Interestingly, the fractional abundance of KMT2A-AF9 fusion transcripts (relative to wild-type transcript abundance) remained stable over the duration of the experiment (FIG. 2C).

### Development and Validation of a Pooled Droplet Digital PCR Assay

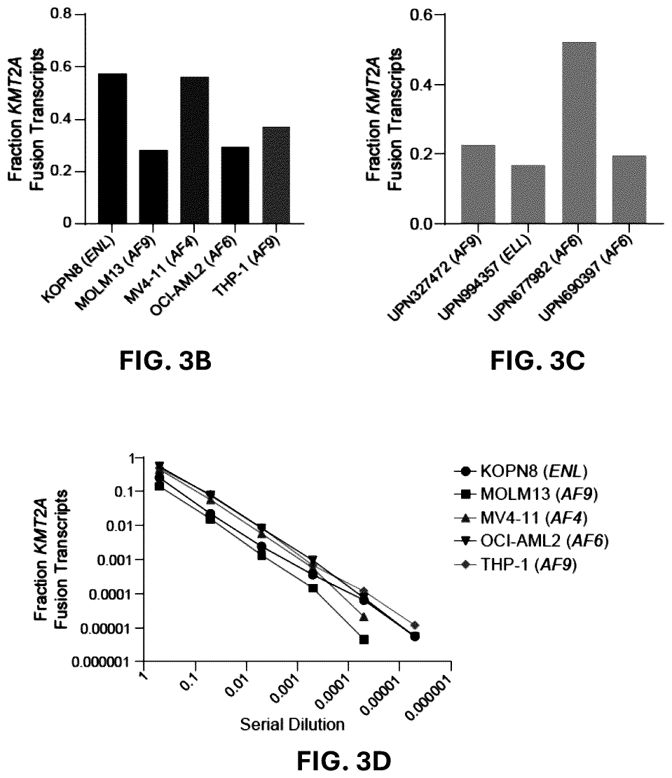

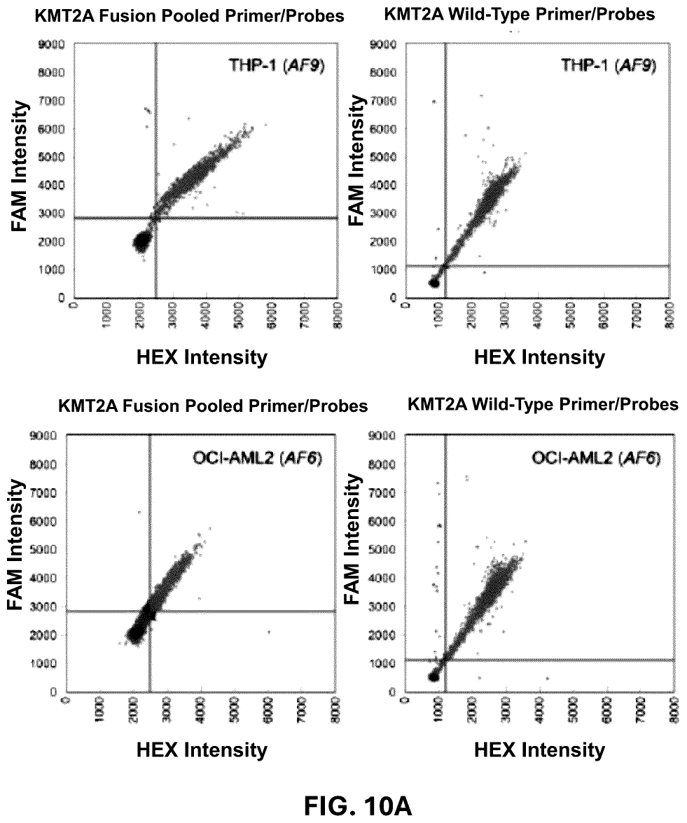

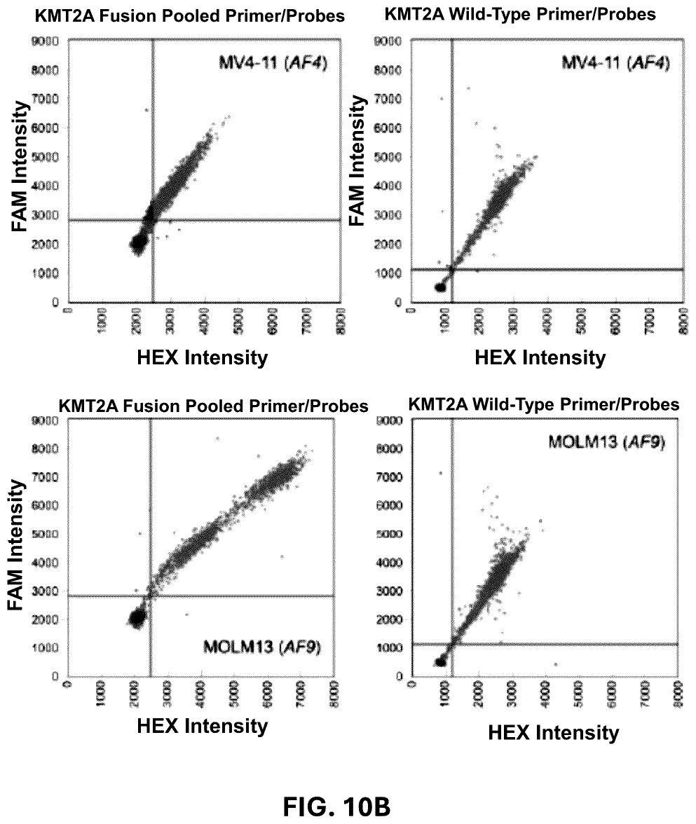

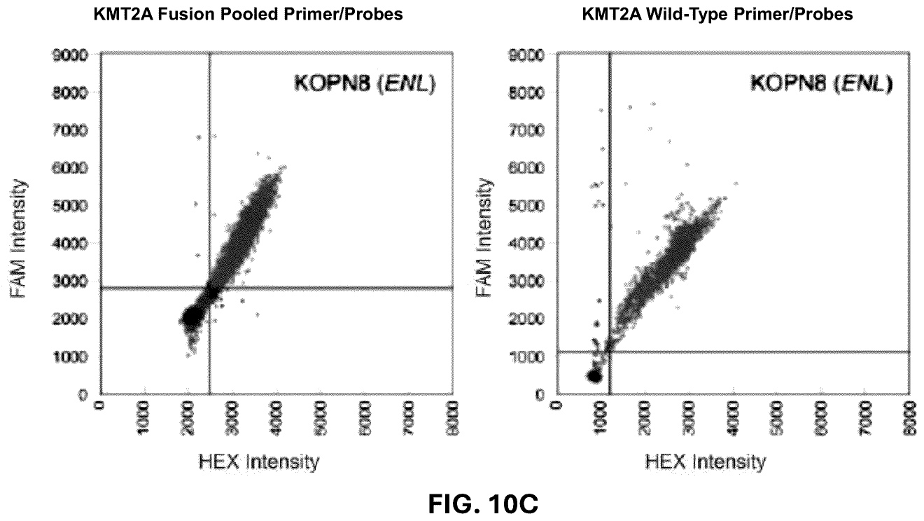

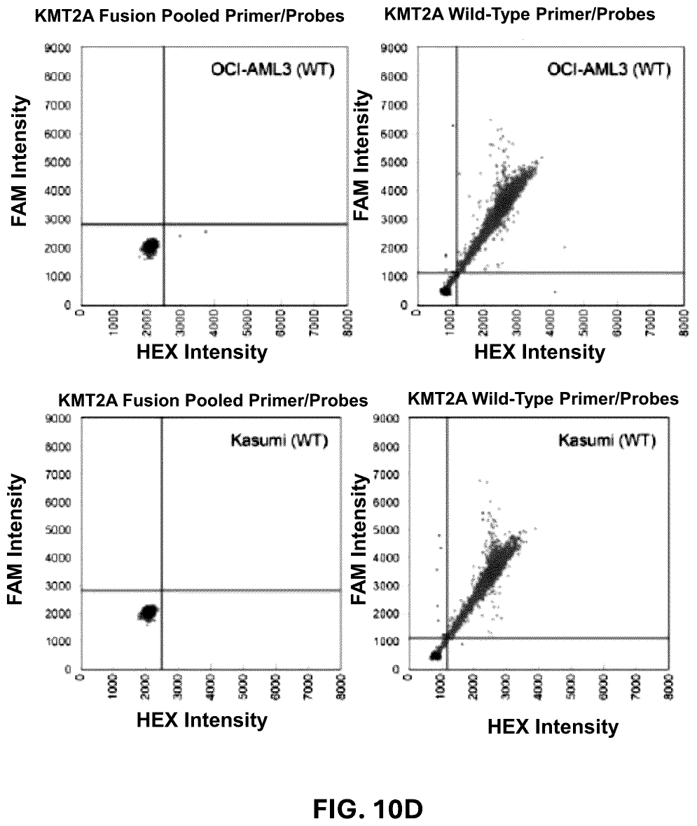

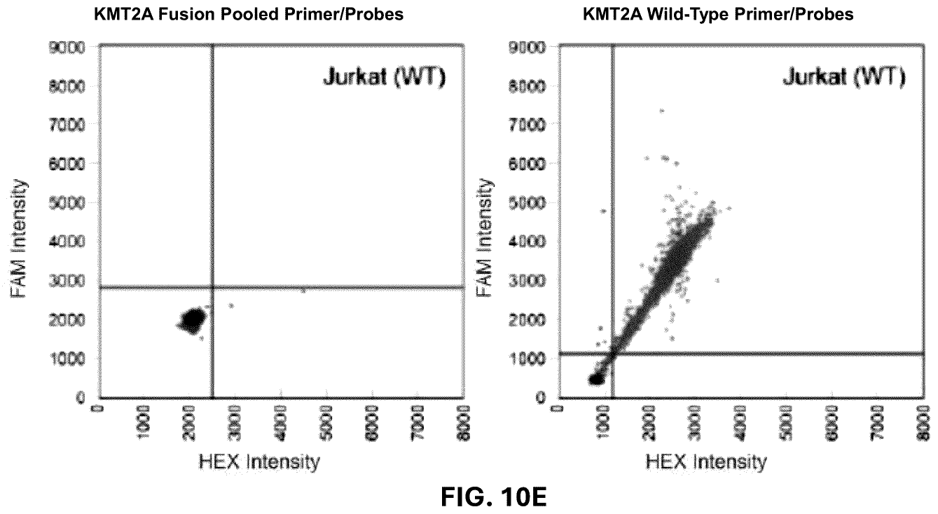

Using a single primer/probe pair for tracking leukemic clones is useful when the oncogenic KMT2A fusion is known. However, in discovery settings when the KMTA2 fusion is unknown, for example when screening for KMT2A fusions in patients receiving TOP2 inhibitors, simultaneous testing for all fusions is necessary. Since each reaction is limited by availability of patient sample RNA, a pooled primer/probe strategy was designed to enable discovery for the five most common KMT2A fusions in the same reaction (FIG. 13, Methods). The pooled reagents detected KMT2A fusions in all cell lines known to harbor KMT2A fusions and found no evidence of KMT2A fusions in cell lines known not to harbor KMT2A fusions (FIG. 3A, FIG. 10). The fraction of KMT2A transcripts originating from a KMT2A fusion was found to be 0.28-0.57 across the cell lines (FIG. 3B).

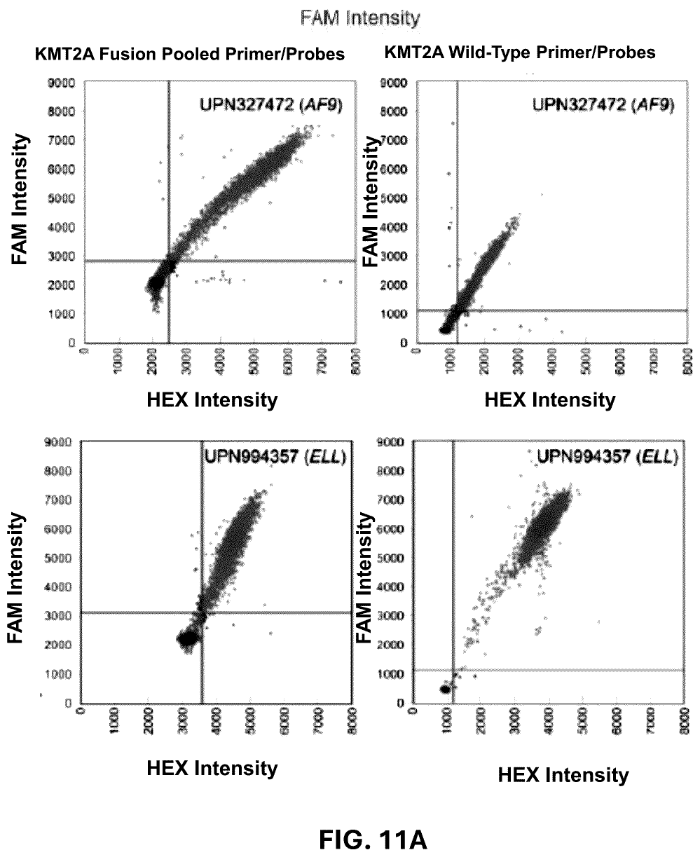

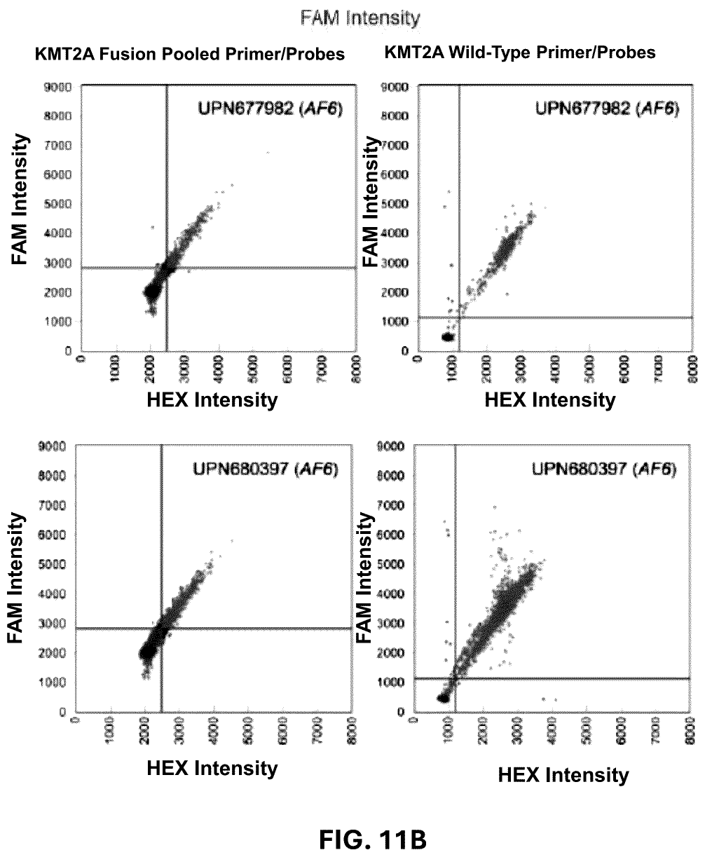

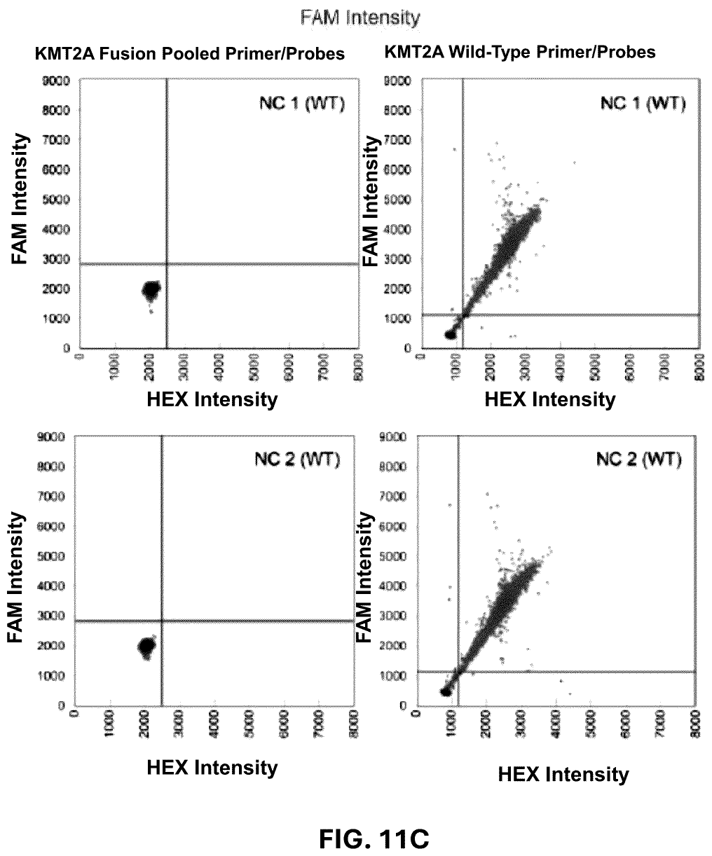

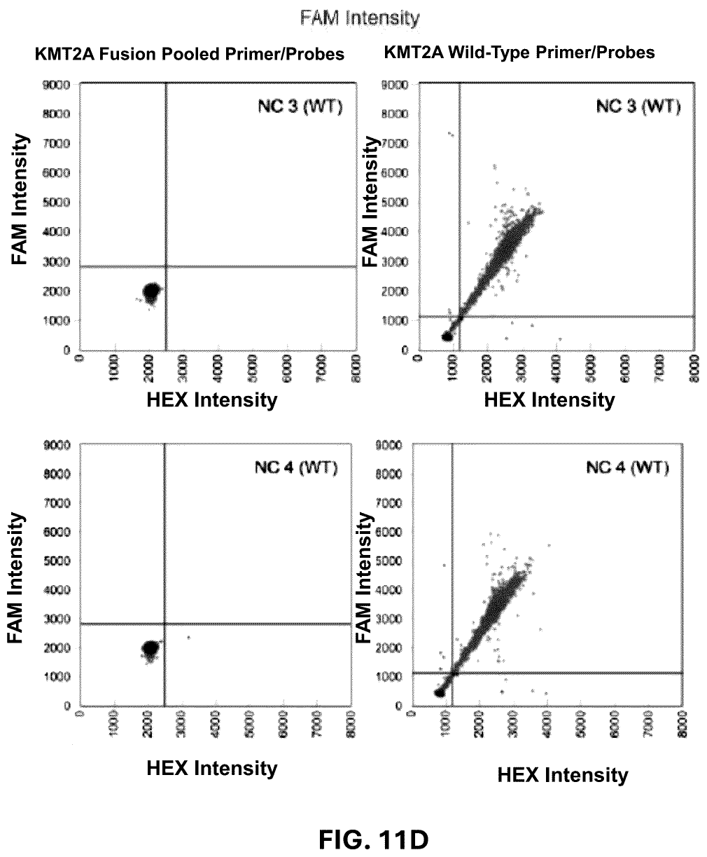

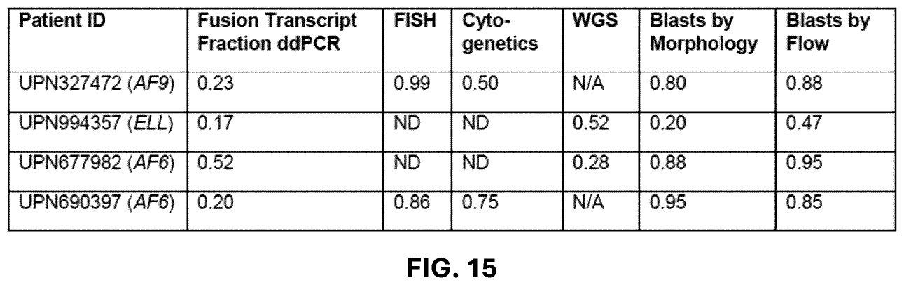

The pooled reagents also detected KMT2A fusions in AML patient samples known to harbor KMT2A fusions (FIG. 11). The transcript abundance of the KMT2A fusion was calculated relative to the wild-type KMT2A fusion abundance and compared to orthogonal metrics of leukemia burden (FIG. 3C, FIG. 15). Four additional healthy control human samples were identified without any known KMT2A fusions, and the pooled primer/probe assay did not find any evidence of KMT2A fusions (FIG. 11).

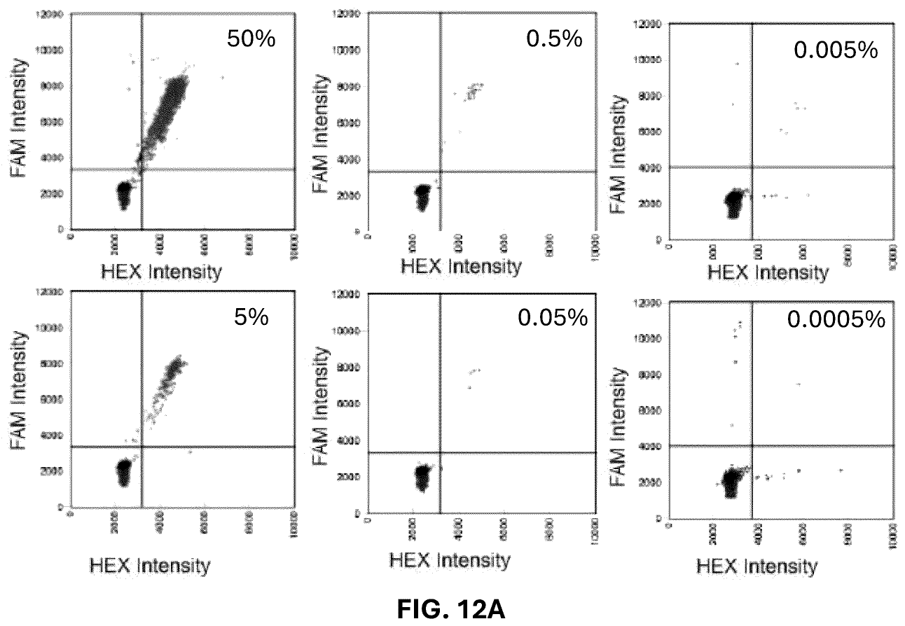

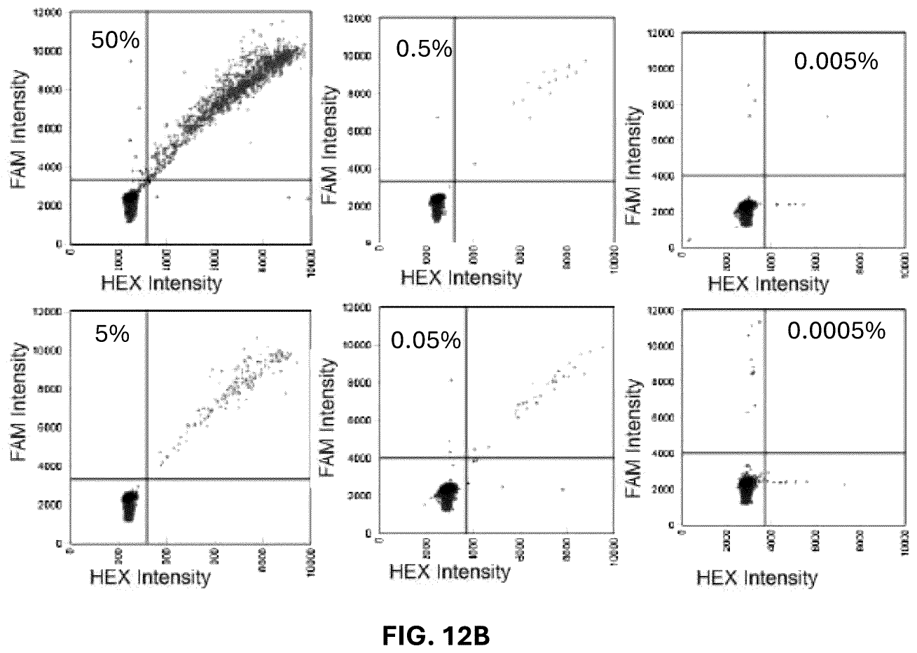

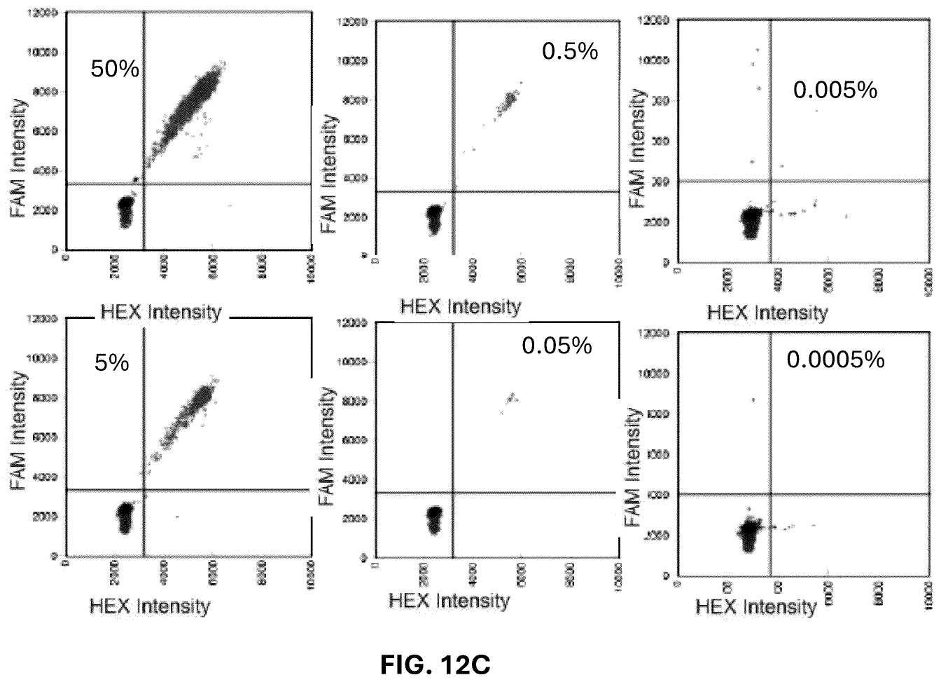

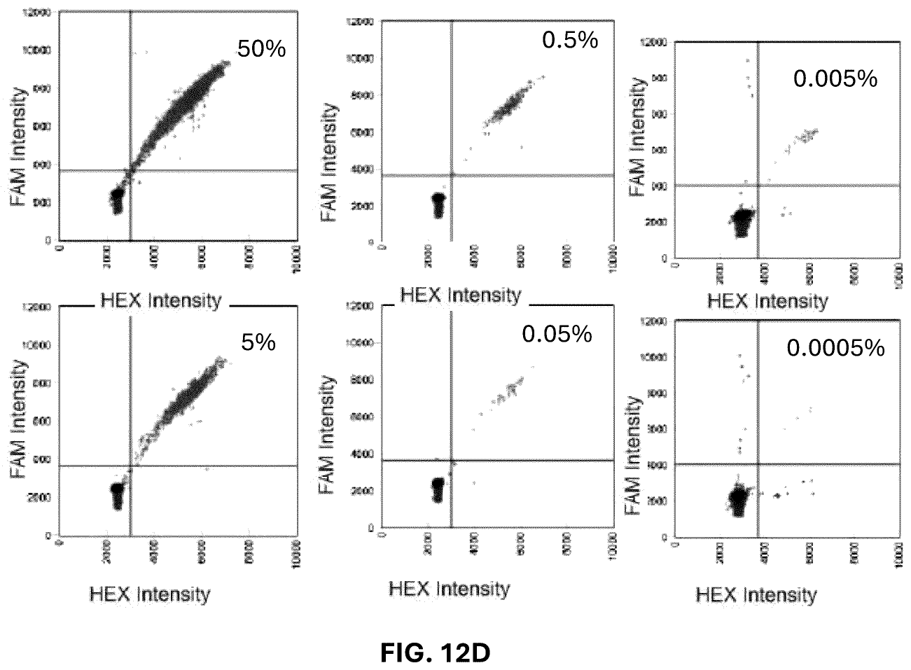

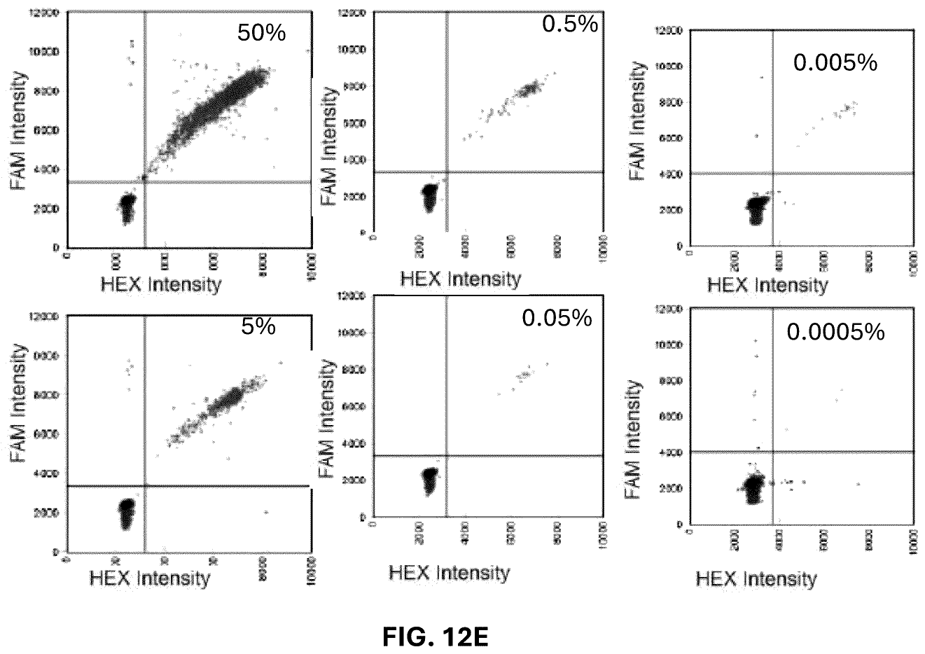

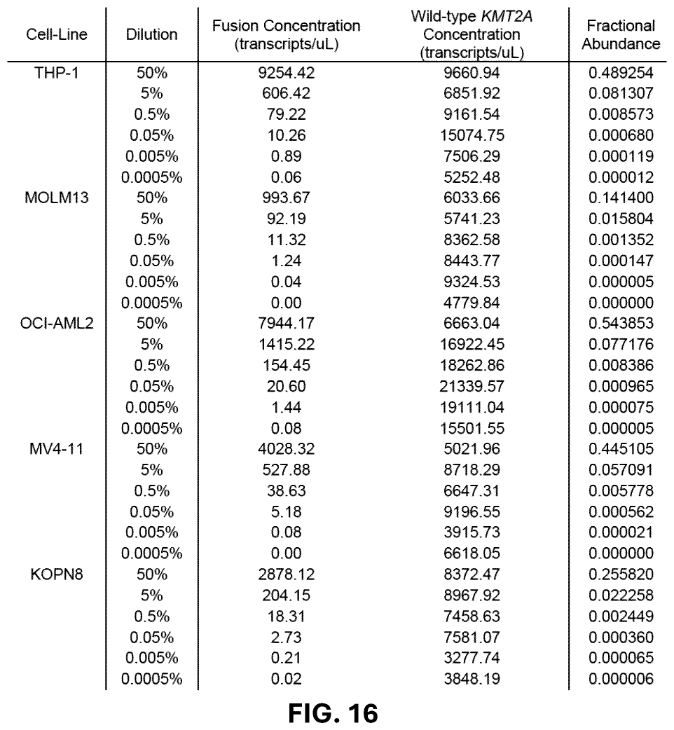

Finally, a cell line dilution series experiment was conducted using the pooled primer/probe reagents. Cell lines known to harbor KMT2A fusions were serially diluted into OCI-AML3 cells (KMT2A wild-type) and KMT2A fusions were detected using the same pooled primer/probe reagent for each cell line dilution experiment (FIG. 12, FIG. 16). The ratio of KMT2A fusion transcript abundance to KMT2A wild-type transcript abundance matched the expected dilution across 5-6 orders of magnitude (FIG. 3D).

### DISCUSSION

The results of these experiments demonstrated a method for KMT2A fusion detection using dual-color ddPCR. The assay was compared to prior techniques using bulk qPCR. Sensitive detection was demonstrated over several logs of dynamic range. The assay was demonstrated to be specific and reliably excluded patient samples and cell lines that did not harbor KMT2A fusions. This assay improved upon existing qPCR strategies due to its ease of use, accurate transcript quantification, ease for multiplex analysis, and flexibility to modify or expand the target panel and reproducibility. The assay did not require standard curves for calibration, as the quantification obtained by ddPCR was absolute. Prior efforts to develop a digital quantification for KMT2A fusions have been limited due to the promiscuous nature of KMT2A fusions with over 80 known KMT2A fusion partners. This assay targeted the five most common KMT2A fusion partners that encompass approximately 80% of KMT2A rearranged AML cases. The limit of detection for this assay was variable based on the amount of input material, but in the dilution experiments, cells harboring KMT2A fusions were reliably identified when their estimated abundance was 10 cells out of 2 million total cells.

This assay sensitively and specifically detected KMT2A fusions by ddPCR, even if limited to the most common fusions. Given the ease of assay development and validation, additional KMT2A fusions could be added to the assay with minimal cost and effort. The assay compared KMT2A fusion abundance to wild-type KMT2A expression, which should have similar gene regulation. This normalization was supported by the cell line dilution experiments that used the ratio of KMT2A fusions to KMT2A wild-type transcript abundance to estimate the fraction of cells harboring KMT2A fusions, which matched abundance at each serial dilution.

What is claimed is:

1. A method for monitoring an abundance of expression of at least one oncogenic KMT2A fusion in a subject, each KMT2A fusion comprising a KMT2A fragment fused at a fusion site to a partner gene fragment, the method comprising:
a. providing:
i. a biological sample from the subject, the sample comprising an amount of RNA;
ii. at least one forward PCR primer targeting a KMT2A start codon of the KMT2A fragment upstream of the fusion site and at least one reverse PCR primer targeting a partner gene start codon of the partner gene fragment downstream of the fusion site;
iii. at least one KMT2A probe comprising a first fluorescent reporter, each KMT2A probe configured to anneal to the KMT2A fragment between the KMT2A start codon and the fusion site; and
iv. at least one partner gene probe comprising a second fluorescent reporter, each partner gene probe configured to anneal to the partner gene fragment between the partner start codon and the fusion site;
b. extracting the amount of RNA from the biological sample and synthesizing an amount of cDNA from the amount of RNA;
c. subjecting a mixture of the amount of cDNA, the at least one forward PCR primer, the at least one reverse PCR primer, the at least one KMT2A probe, and the at least one partner gene probe to droplet digital PCR (ddPCR) to obtain a plurality of paired fluorescence intensities from first and second reporters; and
d. estimating the abundance of expression of the at least one oncogenic KMT2A fusion based on the plurality of paired fluorescence intensities obtained from the ddPCR.

2. The method of claim 1, wherein the first and second fluorescent reporters are independently selected from fluorescein and hexachlorofluorescein, wherein the first fluorescent reporter is different from the second fluorescent reporter.

3. The method of claim 1, wherein the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof.

4. The method of claim 3, wherein the at least one oncogenic KMT2A fusion is one KMT2A fusion from the group consisting of KMT2A-AF9, KMT2A-AF4, KMT2A-AF6, KMT2A-ENL, and KMT2A-ELL.

5. The method of claim 4, wherein:
a. the at least one forward PCR primer targets the KMT2A start codon of the KMT2A fragment selected from KMT2A exon 7, KMT2A exon 9, and any combination thereof; and
b. the at least one reverse PCR primer targets the partner start codon of the partner gene fragment selected from AF9 exon 6, AF4 exon 5, AF6 exon 2, ENL exon 7, ELL exon 3, and any combination thereof.

6. The method of claim 5, wherein:
a. the at least one forward PCR primer comprises a nucleotide sequence selected from SEQ ID NO:1 targeting KMT2A exon 7, SEQ ID NO:2 targeting KMT2A exon 9, and any combination thereof; and
b. the at least one reverse PCR primer comprises a nucleotide sequence selected from:
i. SEQ ID NO:7 targeting AF9 exon 6;
ii. SEQ ID NO:5 targeting AF4 exon 5;
iii. SEQ ID NO:9 targeting AF6 exon 2;
iv. SEQ ID NO:13 targeting ENL exon 7;
vi. any combination thereof.

7. The method of claim 5, wherein the at least one KMT2A probe comprises a nucleotide sequence selected from SEQ ID NO:16 targeting KMT2A exon 7, SEQ ID NO:22 targeting KMT2A exon 9, and any combination thereof.

8. The method of claim 5, wherein the at least one partner gene probe comprises a nucleotide sequence selected from:
a. SEQ ID NO:18 targeting AF9 exon 6;
b. SEQ ID NO:17 targeting AF4 exon 5;
c. SEQ ID NO:19 targeting AF6 exon 2;
d. SEQ ID NO:21 targeting ENL exon 7;
e. SEQ ID NO:23 targeting ELL exon 3; and
f. any combination thereof.

9. The method of claim 1, wherein the sample comprises at least one of a peripheral blood sample, a bone marrow sample, a solid tumor sample, and any combination thereof.

10. The method of claim 1, further comprising identifying at least one subject-specific KMT2A fusion from a nucleic acid sequencing of the subject's cancer, wherein each subject-specific KMT2A fusion comprises a KMT2A fragment fused at a fusion site to a subject-specific partner gene fragment, wherein:
a. the at least one reverse PCR primer further comprises an additional reverse PCR primer targeting a subject-specific partner gene start codon of the subject-specific partner gene fragment downstream of the fusion site; and
b. the at least one partner gene probe further includes at least one subject-specific partner gene probe comprising the second fluorescent reporter, wherein each subject-specific partner gene probe is configured to anneal to the subject-specific partner gene fragment between the subject-specific partner gene start codon and the fusion site.

11. The method of claim 1, further comprising classifying the cancer patient as having measurable residual disease if the abundance of expression of the at least one oncogenic KMT2A fusion falls above a threshold value of 0.001% as measured by ddPCR for the oncogenic KMT2A fusion.

12. The method of claim 1, further comprising:
a. providing first and second biological samples from the subject, wherein the second sample is obtained after administration of a treatment to the subject;
b. estimating a first and second abundance of expression of the at least one oncogenic KMT2A fusion based on the first and second biological samples;
c. estimating an efficacy of the treatment, a development of treatment-related oncogenic KMT2A fusions, a prognosis, and any combination thereof based on changes between the first and second abundances of expression of the at least one oncogenic KMT2A fusion.

13. A method for personalized monitoring an abundance of expression of at least one oncogenic KMT2A fusion in a patient, each KMT2A fusion comprising a KMT2A fragment fused at a fusion site to a partner gene fragment, the method comprising:
a. providing an initial sequencing of the patient's leukemia at a nucleotide-level resolution;
b. identifying at least one oncogenic KMT2A fusion based on the initial sequencing;
c. providing:
i. a biological sample from the patient, the sample comprising an amount of RNA;
ii. at least one forward PCR primer targeting a KMT2A start codon of the KMT2A fragment upstream of the fusion site and at least one reverse PCR primer targeting a partner gene start codon of the partner gene fragment downstream of the fusion site;
iii. at least one KMT2A probe comprising a first fluorescent reporter, each KMT2A probe configured to anneal to the KMT2A fragment between the KMT2A start codon and the fusion site; and
iv. at least one partner gene probe comprising a second fluorescent reporter, each partner gene probe configured to anneal to the partner gene fragment between the partner start codon and the fusion site;
d. extracting the amount of RNA from the biological sample and synthesizing an amount of cDNA from the amount of RNA;
e. subjecting a mixture of the amount of cDNA, the at least one forward PCR primer, the at least one reverse PCR primer, the at least one KMT2A probe, and the at least one partner gene probe to droplet digital PCR (ddPCR) to obtain a plurality of paired fluorescence intensities from first and second reporters; and
f. estimating the abundance of expression of the at least one oncogenic KMT2A fusion based on the plurality of paired fluorescence intensities obtained from the ddPCR.

14. An assay to monitor an abundance of expression of at least one oncogenic KMT2A fusion in a subject, each KMT2A fusion comprising a KMT2A fragment fused at a fusion site to a partner gene fragment, the assay comprising:
a. at least one forward PCR primer targeting a KMT2A start codon of the KMT2A fragment upstream of the fusion site and at least one reverse PCR primer targeting a partner gene start codon of the partner gene fragment downstream of the fusion site;
b. at least one KMT2A probe comprising a first fluorescent reporter, each KMT2A probe configured to anneal to the KMT2A fragment between the KMT2A start codon and the fusion site; and
c. at least one partner gene probe comprising a second fluorescent reporter, each partner gene probe configured to anneal to the partner gene fragment between the partner start codon and the fusion site;
wherein the at least one forward PCR primer, the at least one reverse PCR primer, the at least one KMT2A probe, and the at least one partner gene probe are combined with an amount of cDNA synthesized from an amount of RNA from the subject to form a mixture for processing in a droplet digital PCR (ddPCR) device.

15. The assay of claim 14, wherein the first and second fluorescent reporters are independently selected from fluorescein and hexachlorofluorescein, wherein the first fluorescent reporter is different from the second fluorescent reporter.

16. The assay of claim 14, wherein the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof.

17. The assay of claim 16, wherein the at least one oncogenic KMT2A fusion is one KMT2A fusion from the group consisting of KMT2A-AF9, KMT2A-AF4, KMT2A-AF6, KMT2A-ENL, and KMT2A-ELL.

18. The assay of claim 17, wherein:
a. the at least one forward PCR primer targets the KMT2A start codon of the KMT2A fragment selected from KMT2A exon 7, KMT2A exon 9, and any combination thereof; and
b. the at least one reverse PCR primer targets the partner start codon of the partner gene fragment selected from AF9 exon 6, AF4 exon 5, AF6 exon 2, ENL exon 7, ELL exon 3, and any combination thereof.

19. The assay of claim 18, wherein:
a. the at least one forward PCR primer comprises a nucleotide sequence selected from SEQ ID NO:1 targeting KMT2A exon 7, SEQ ID NO:2 targeting KMT2A exon 9, and any combination thereof; and
b. the at least one reverse PCR primer comprises a nucleotide sequence selected from:
i. SEQ ID NO:7 targeting AF9 exon 6;
ii. SEQ ID NO:5 targeting AF4 exon 5;
iii. SEQ ID NO:9 targeting AF6 exon 2;
iv. SEQ ID NO:13 targeting ENL exon 7;
v. SEQ ID NO:14 targeting ELL exon 3; and
vi. any combination thereof.

20. The assay of claim 18, wherein the at least one KMT2A probe comprises a nucleotide sequence selected from SEQ ID NO:16 targeting KMT2A exon 7, SEQ ID NO:22 targeting KMT2A exon 9, and any combination thereof.

21. The assay of claim 18, wherein the at least one partner gene probe comprises a nucleotide sequence selected from:
a. SEQ ID NO:18 targeting AF9 exon 6;
b. SEQ ID NO:17 targeting AF4 exon 5;
c. SEQ ID NO:19 targeting AF6 exon 2;
d. SEQ ID NO:21 targeting ENL exon 7;
e. SEQ ID NO:23 targeting ELL exon 3; and
f. any combination thereof.

22. A kit for monitoring an abundance of expression of at least one oncogenic KMT2A fusion in a subject, each KMT2A fusion comprising a KMT2A fragment fused at a fusion site to a partner gene fragment, the kit comprising:
a. at least one forward PCR primer targeting a KMT2A start codon of the KMT2A fragment upstream of the fusion site and at least one reverse PCR primer targeting a partner gene start codon of the partner gene fragment downstream of the fusion site;
b. at least one KMT2A probe comprising a first fluorescent reporter, each KMT2A probe configured to anneal to the KMT2A fragment between the KMT2A start codon and the fusion site; and
c. at least one partner gene probe comprising a second fluorescent reporter, each partner gene probe configured to anneal to the partner gene fragment between the partner start codon and the fusion site.

23. The kit of claim 22, wherein the first and second fluorescent reporters are independently selected from fluorescein and hexachlorofluorescein, wherein the first fluorescent reporter is different from the second fluorescent reporter.

24. The kit of claim 22, wherein the at least one oncogenic KMT2A fusion is selected from KMT2A-AF9, KMT2A-AF4, KMT2A-AF6, KMT2A-ENL, KMT2A-ELL, at least one subject-specific KMT2A fusion and any combination thereof.

25. The kit of claim 24, wherein the at least one oncogenic KMT2A fusion is one KMT2A fusion from the group consisting of KMT2A-AF9, KMT2A-AF4, KMT2A-AF6, KMT2A-ENL, and KMT2A-ELL.

26. The kit of claim 24, wherein:
a. the at least one forward PCR primer targets the KMT2A start codon of the KMT2A fragment selected from KMT2A exon 7, KMT2A exon 9, and any combination thereof; and
b. the at least one reverse PCR primer targets the partner start codon of the partner gene fragment selected from AF9 exon 6, AF4 exon 5, AF6 exon 2, ENL exon 7, ELL exon 3, and any combination thereof.

27. The kit of claim 26, wherein:
a. the at least one forward PCR primer comprises a nucleotide sequence selected from SEQ ID NO:1 targeting KMT2A exon 7, SEQ ID NO:2 targeting KMT2A exon 9, and any combination thereof; and
b. the at least one reverse PCR primer comprises a nucleotide sequence selected from:
i. SEQ ID NO:7 targeting AF9 exon 6;
ii. SEQ ID NO:5 targeting AF4 exon 5;
iii. SEQ ID NO:9 targeting AF6 exon 2;
iv. SEQ ID NO:13 targeting ENL exon 7;
vi. any combination thereof.

28. The kit of claim 26, wherein the at least one KMT2A probe comprises a nucleotide sequence selected from SEQ ID NO:16 targeting KMT2A exon 7, SEQ ID NO:22 targeting KMT2A exon 9, and any combination thereof.

29. The kit of claim 26, wherein the at least one partner gene probe comprises a nucleotide sequence selected from:
a. SEQ ID NO:18 targeting AF9 exon 6;
b. SEQ ID NO:17 targeting AF4 exon 5;
c. SEQ ID NO:19 targeting AF6 exon 2;
d. SEQ ID NO:21 targeting ENL exon 7;
e. SEQ ID NO:23 targeting ELL exon 3; and
f. any combination thereof.


SEQUENCE LISTING (25 sequences)

SEQ ID NO: 1 - DNA, 20 bp, synthetic construct
  source 1..20: mol_type = other DNA
ccactccagcttccaggaag

SEQ ID NO: 2 - DNA, 20 bp, synthetic construct
  source 1..20: mol_type = other DNA
cagcactctctccaatggca

SEQ ID NO: 3 - DNA, 20 bp, synthetic construct
  source 1..20: mol_type = other DNA
tgccattggagagagtgctg

SEQ ID NO: 4 - DNA, 20 bp, synthetic construct
  source 1..20: mol_type = other DNA
tttgcaacgacgacaacacc

SEQ ID NO: 5 - DNA, 20 bp, synthetic construct
  source 1..20: mol_type = other DNA
aacttggatggctcagctgt

SEQ ID NO: 6 - DNA, 20 bp, synthetic construct
  source 1..20: mol_type = other DNA
ttctgactctgcactgctgg

SEQ ID NO: 7 - DNA, 20 bp, synthetic construct
  source 1..20: mol_type = other DNA
cttgttgcctggtctgggat

SEQ ID NO: 8 - DNA, 20 bp, synthetic construct
  source 1..20: mol_type = other DNA
tgctttcactatcgctgcca

SEQ ID NO: 9 - DNA, 20 bp, synthetic construct
  source 1..20: mol_type = other DNA
gtcgaaatttctccgcgagc

SEQ ID NO: 10 - DNA, 20 bp, synthetic construct
  source 1..20: mol_type = other DNA
ggagaggacagcattcgcat

SEQ ID NO: 11 - DNA, 20 bp, synthetic construct
  source 1..20: mol_type = other DNA
cagtcgtgagtgaacccctc

SEQ ID NO: 12 - DNA, 20 bp, synthetic construct
  source 1..20: mol_type = other DNA
ggtcgtaggtgaagcagacc

SEQ ID NO: 13 - DNA, 20 bp, synthetic construct
  source 1..20: mol_type = other DNA
gttctgggatggctcgaagt

SEQ ID NO: 14 - DNA, 20 bp, synthetic construct
  source 1..20: mol_type = other DNA
ggccgatgttggagaggtag

SEQ ID NO: 15 - DNA, 20 bp, synthetic construct
  source 1..20: mol_type = other DNA
cagtccaggtgaacttcccc

SEQ ID NO: 16 - DNA, 20 bp, synthetic construct
  source 1..20: mol_type = other DNA
agcaggtctcccagccagca

SEQ ID NO: 17 - DNA, 24 bp, synthetic construct
  source 1..24: mol_type = other DNA
acccattcatggccgcctcctttg

SEQ ID NO: 18 - DNA, 20 bp, synthetic construct
  source 1..20: mol_type = other DNA
cctgccagctccagctccag

SEQ ID NO: 19 - DNA, 26 bp, synthetic construct
  source 1..26: mol_type = other DNA
tcgggtctctagtactgccaccactc

SEQ ID NO: 20 - DNA, 24 bp, synthetic construct
  source 1..24: mol_type = other DNA
accacctccggtcaataagcagga

SEQ ID NO: 21 - DNA, 20 bp, synthetic construct
  source 1..20: mol_type = other DNA
agtctgagctggagtctgag

SEQ ID NO: 22 - DNA, 27 bp, synthetic construct
  source 1..27: mol_type = other DNA
cagcagatggagtccacaggatcagag

SEQ ID NO: 23 - DNA, 18 bp, synthetic construct
  source 1..18: mol_type = other DNA
aacgtccgcgcctctgcg

SEQ ID NO: 24 - RNA, 20 nt, synthetic construct
  source 1..20: mol_type = other RNA
ttagaaatggaggctgggcg

SEQ ID NO: 25 - RNA, 20 nt, synthetic construct
  source 1..20: mol_type = other RNA
atctactttgcctgcgctat